(19) United States
(12) Patent Application Publication
(10) Pub. No.: US 2026/0020775 A1
(43) Pub. Date: Jan. 22, 2026

(54) DATA LOGGING RESPIRATORY SENSOR INTEGRATION BLOCK

(71) Applicant: Texas Tech University System, Lubbock, TX (US)

(72) Inventors: Shelby Lacouture, Levelland, TX (US); Annette L. Sobel, Lubbock, TX (US); Robert V. Duncan, Lubbock, TX (US); Cuikun Lin, Lubbock, TX (US); Laszlo Hunyadi, Amarillo, TX (US); Emily Sundman, Amarillo, TX (US); Noah Plues, Lubbock, TX (US); Mitchell Kelley, Lubbock, TX (US)

(21) Appl. No.: 19/289,977

(22) Filed: Aug. 4, 2025

Related U.S. Application Data
(63) Continuation-in-part of application No. 18/860,033.
(60) Provisional application No. 63/334,254, filed on Apr. 25, 2022.

(51) Int. Cl. A61B 5/087; A61B 5/08; G16H 50/20

(52) U.S. Cl. CPC A61B 5/087; A61B 5/0803; G16H 50/20

(57) ABSTRACT

The present invention includes a Data-Logging Sensor Integration Block (DLSIB) system for real-time respiratory data for use with humans and animals comprising at least one chamber comprising an interior, an inlet and outlet for air, wherein two or more sensors are in fluid communication with the interior of the at least one chamber, wherein the two or more sensors are selected from a nitric oxide sensor, a nitrous oxide sensor an oxygen sensor, an ozone sensor, a carbon dioxide sensor, a carbon monoxide sensor, a pressure sensor, a temperature sensor, a humidity sensor a mass flow rate sensor, an SpO₂ sensor, or a PaCO₂ sensor; and a processor connected to each of the two or more sensors, and wherein the DLSIB system measures in real-time or internally records outputs from the two or more sensors for each breath of a subject.

### CROSS-REFERENCE TO RELATED APPLICATIONS

This application is a continuation-in-part of U.S. National Phase application Ser. No. 18/860,033 filed on Oct. 25, 2024, which claims priority to PCT International Application Serial No. PCT/US23/065831 filed Apr. 17, 2023, and Provisional Patent Application Ser. No. 63/334,254 filed Apr. 25, 2022, the entire contents of each of which are incorporated herein by reference.

### TECHNICAL FIELD OF THE INVENTION

The present invention relates in general to the field of respirators or breathing devices, and more particularly, to a modular data logging respiratory sensor integration block system.

### STATEMENT OF FEDERALLY FUNDED RESEARCH

None.

### BACKGROUND OF THE INVENTION

Without limiting the scope of the invention, its background is described in connection with respirators or breathing devices, diagnostic equipment, and both medical and home “health monitoring”.

Respiratory assist devices help patients in need of support for breathing, removal of carbon dioxide, and therapy to reduce atrophy of abdominal wall muscles. Demand for these devices rose significantly during the COVID-19 pandemic as they are invaluable for treating patients severely impacted by the pandemic. Mechanical ventilation is required when it becomes very difficult for a patient to breathe or get enough oxygen into their blood, which indicates a patient might be experiencing respiratory failure. Mechanical ventilators are medical devices that move air in and out of the lungs to keep the patient alive. Some ventilators provide support to patients who do not require complex critical care ventilators and these typically consist of a flexible breathing circuit, a control system, monitors, and alarms.

Other systems might also consist of oxygen accumulators, heated humidifiers, or heat and moisture exchangers to improve patient comfort. Long-term and emergency care devices use positive pressure to deliver gas to the lungs at normal breathing rates and tidal volumes through an endotracheal tube, a tracheostomy cannula, or a mask. Another assistive respiratory device is the continuous positive airway pressure therapy (CPAP), which uses a machine to treat patients suffering from obstructive sleep apnea (OSA). A CPAP machine increases air pressure in the throat so that the airway doesn't collapse during inhalation.

Asthma is a long-term respiratory condition that affects individuals of all ages, as well as certain animals such as horses and cats. It is characterized by inflammation and constriction of the airways, leading to difficulties in breathing. Symptoms commonly include coughing, wheezing, shortness of breath, and chest tightness. These symptoms can vary in intensity and frequency over time.

In the case of humans, the global market for asthma treatment witnessed significant growth, with a value of USD 25.8 billion in 2021. It is projected to reach USD 30.1 billion by 2030, exhibiting a Compound Annual Growth Rate (CAGR) of 2.6% from 2022 to 2030. Prominent players in this market include GSK, AstraZeneca, Sanofi-Aventis, Bochringer Ingelheim, and Teva. These companies have focused on product development and obtaining regulatory approvals. They offer a range of asthma medications and related accessories like inhalers.

Regarding veterinary medicine, the global market size was estimated to be USD 44.59 billion in 2022, and it is anticipated to grow at a lucrative Compound Annual Growth Rate (CAGR) of 8.2% during the forecast period. The market's expansion is primarily driven by the increasing livestock population and production in developing countries. Additionally, the rise in zoonotic and chronic diseases, coupled with the growing consumption of meat and rising awareness about food-borne illnesses, contribute to the market's growth.

Although respiratory support devices have advanced significantly over the last few decades, there are still aspects of the systems that need improvement. Respiratory monitoring is important and could be significantly enhanced by advances in noninvasive monitoring of blood gases, as well as monitoring of brain and organ oxygenation, perfusion, and hemodynamics. Noninvasive methods to assess lung volume and perfusion shows promise but have been limited to complex techniques that have been primarily used in research studies or invasive clinical procedures.

### SUMMARY OF THE INVENTION

As embodied and broadly described herein, an aspect of the present disclosure relates to a Data-Logging Sensor Integration Block (DLSIB) system for real-time respiratory data for use with humans and animals comprising: at least one chamber comprising an interior, an inlet and outlet for air, wherein two or more sensors are in fluid communication with the interior of the at least one chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor; an SpO₂ sensor, or a PaCO₂ sensor; and a processor connected to each of the two or more sensors, and wherein the DLSIB system measures in real-time or internally records respiratory pressure and the inhalation and exhalation volumes of each breath of a subject. In one aspect, at least one of: a partial pressure of NO, O₂, and CO₂ during the breath, the integrated quantity of oxygen added to the blood, and the integrated quantity of CO₂ removed from the blood during each breath, are determined. In another aspect, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, or inhalation of soot from fires, vog from volcanic eruptions, dust from burn pits, and/or dust inside mining shafts. In another aspect, the DLSIB system detection of physiological and physical obstructions that might be preventing optimal oxygen-carbon dioxide exchange at the alveolar level, which can expedite diagnostic and therapeutic pulmonary hygienic intervention. In another aspect, the DLSIB system is connected between an air pump or respirator and a mask; is integral with a mask; or is integral with an air pump or respirator. In another aspect, the system further comprises a display connected to the processor, wherein the display shows subject data in an aggregated or disaggregated graphic. In another aspect, the processor and two or more sensors are wired or wireless. In another aspect, an input and output of the at least one chamber each connect to an input and an output hose or at least chamber, respectively. In another aspect, the system comprises, consists essentially of, or consists of: the nitric oxide sensor, the nitrous oxide sensor, the oxygen sensor, the ozone sensor, the carbon dioxide sensor, the carbon monoxide sensor, the pressure sensor, the temperature sensor, the humidity sensor, the ammonia sensor, the mass flow rate sensor, the SpO₂ sensor, and the PaCO₂ sensor.

As embodied and broadly described herein, an aspect of the present disclosure relates to a method of obtaining real-time respiratory data comprising: providing a Data-Logging Sensor Integration Block (DLSIB) system capable of connecting to a respiratory system of a subject for obtaining the real-time respiratory data, the device comprising: at least one chamber comprising an interior, an inlet and outlet for air, wherein two or more sensors are in fluid communication with the interior of the at least chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor, an SpO₂ sensor, or a PaCO₂ sensor; and a processor connected to each of the two or more sensors, and wherein the DLSIB system measures in real-time respiratory pressure and the inhalation and exhalation volumes of each breath of a subject; and calculating the real-time respiratory data with the processor. In one aspect, at least one of: a partial pressure of NO, O₂, and CO₂ during the breath, the integrated quantity of oxygen added to the blood, and the integrated quantity of CO₂ removed from the blood during each breath, are determined. In another aspect, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, inhalation of soot from fires, vog from volcanic eruptions, dust from burn pits, and/or dust inside mining shafts. In another aspect, the DLSIB system is configured for use with an animal, mammal, or human.

In another aspect, the DLSIB system enables detection of physiological and physical obstructions that might be preventing optimal oxygen-carbon dioxide exchange at the alveolar level, which can expedite diagnostic and therapeutic pulmonary hygienic intervention. In another aspect, the DLSIB system can be connected between an air pump or respirator and a mask; is integral with a mask; or is integral with an air pump or respirator. In another aspect, the system further comprises providing a display connected to the processor, wherein the display shows subject data in an aggregated or disaggregated graphic. In another aspect, the processor and the two or more sensors are wired or wireless. In another aspect, an input and output of the at least one chamber each connect to an input and an output hose or at least one chamber, respectively. In another aspect, the system comprises, consists essentially of, or consists of: the nitric oxide sensor, the nitrous oxide sensor, the oxygen sensor, the ozone sensor, the carbon dioxide sensor, the carbon monoxide sensor, the pressure sensor, the temperature sensor, the humidity sensor, the ammonia sensor, the mass flow rate sensor, the SpO₂ sensor, and the PaCO₂ sensor. In another aspect, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, inhalation of soot from fires, volcanic smog (vog) from volcanic eruptions, dust from burn pits, and/or dust inside mining shafts. In another aspect, the DLSIB system is configured for use with an animal, mammal, or human.

As embodied and broadly described herein, an aspect of the present disclosure relates to a method of determining the effectiveness of a pulmonary therapy, the method comprising: (a) measuring real-time respiratory data comprising: providing a device capable of connecting to a subject for obtaining the real-time respiratory data, the device comprising: at least one chamber comprising an interior, an inlet and outlet for air, wherein two or more sensors are in fluid communication with the interior of the at least one chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor, an SpO₂ sensor, or a PaCO₂ sensor; and a processor connected to each of the two or more sensors, and wherein the DLSIB system measures in real-time respiratory pressure and the inhalation and exhalation volumes of each breath of a subject; and calculating the real-time respiratory data with the processor; (b) administering a candidate drug to a first subset of the subjects, and a placebo to a second subset of the subjects; (c) generating the real-time respiratory data from the first and second subset of subjects; (d) calculating a difference between the real-time respiratory data in the first and second subset of subjects; and (e) if real-time respiratory data differs between the first and second subset of subjects then calculating an effectiveness of the pulmonary therapy. In another aspect, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, inhalation of soot from fires, volcanic smog (vog) from volcanic eruptions, dust from burn pits, and/or dust inside mining shafts. In another aspect, the DLSIB system is configured for use with an animal, mammal, or human.

As embodied and broadly described herein, an aspect of the present disclosure relates to a method of measuring respiratory function, the method comprising: connecting a device capable of connecting to a subject for obtaining the real-time respiratory data, the device comprising: a mask or at least one chamber comprising an interior, an inlet and outlet for air, wherein two or more sensors are in fluid communication with the interior of the mask or at least one chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor, an SpO₂ sensor, or a PaCO₂ sensor; and a processor connected to each of the two or more sensors, and wherein the device measures in real-time respiratory pressure and the inhalation and exhalation volumes of each breath of a subject; and calculating the real-time respiratory data with the processor to determine respiratory function. In another aspect, the DLSIB system is configured for use with an animal, mammal, or human.

As embodied and broadly described herein, an aspect of the present disclosure relates to a sensor integrated block (SIB) system for real-time respiratory data comprising: a chamber comprising an interior, an inlet and outlet for air, wherein two or more sensors are in fluid communication with the interior of the chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, an ammonia sensor, and a temperature sensor, a humidity sensor; one or more second sensors selected from an SpO₂ sensor or a PaCO₂ sensor; and a processor connected to each of the first and second sensors, and wherein the SIB system measures in real-time respiratory pressure and the inhalation and exhalation volumes of each breath of a subject. In one aspect, the partial pressure of O₂ and CO₂ during the breath, the integrated quantity of oxygen added to the blood, and the integrated quantity of CO₂ removed from the blood during each breath are determined. In another aspect, the SIB system analyzes, displays, and reports subject data to enable a provider to expedite diagnostic and therapeutic decisions and medical evaluations in real-time. In another aspect, the SIB system enables detection of physiologic and physical obstructions that might be preventing optimal oxygen-carbon dioxide exchange at the alveolar level, which can expedite diagnostic and therapeutic pulmonary hygienic intervention. In another aspect, the SIB system can be connected between an air pump or respirator and a mask. In another aspect, the SIB system chamber is integral with a mask. In another aspect, the SIB system chamber is integral with an air pump or respirator. In another aspect, the system further comprises a display connected to the processor, wherein the display shows subject data in an aggregated or disaggregated graphic. In another aspect, the processor and the first, the second, or both the first and second sensors are wired or wireless. In another aspect, the input and output of the chamber each connect to an input and an output hose, respectively. In another aspect, the sensors comprise 3 or 4 of the sensors.

As embodied and broadly described herein, an aspect of the present disclosure relates to a method of obtaining real-time respiratory data comprising: providing a device capable of connecting to a subject for obtaining the real-time respiratory data, the device comprising: a chamber comprising an interior, an inlet and outlet for air, wherein two or more sensors are in fluid communication with the interior of the chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, an ammonia sensor, and a humidity sensor; and one or more second sensors selected from an SpO₂ sensor or a PaCO₂ sensor; and a processor connected to each of the first and second sensors, and wherein the SIB system measures in real-time respiratory pressure and the inhalation and exhalation volumes of each breath of a subject; and calculating the real-time respiratory data with the processor. In one aspect, the partial pressure of O₂ and CO₂ during the breath, the integrated quantity of oxygen added to the blood, and the integrated quantity of CO₂ removed from the blood during each breath are determined. In another aspect, the SIB system analyzes, displays, and reports subject data to enable a provider to expedite diagnostic and therapeutic decisions and medical evaluations in real-time. In another aspect, the SIB system enables detection of physiologic and physical obstructions that might be preventing optimal oxygen-carbon dioxide exchange at the alveolar level, which can expedite diagnostic and therapeutic pulmonary hygienic intervention. In another aspect, the SIB system can be connected between an air pump or respirator and a mask. In another aspect, the SIB system chamber is integral with a mask. In another aspect, the SIB system chamber is integral with an air pump or respirator. In another aspect, the method further comprises providing a display connected to the processor, wherein the display shows subject data in an aggregated or disaggregated graphic. In another aspect, the processor and the first, the second, or both the first and second sensors are wired or wireless. In another aspect, the input and output of the chamber each connect to an input and an output hose, respectively. In another aspect, the sensors comprise 3 or 4 of the sensors.

As embodied and broadly described herein, an aspect of the present disclosure relates to a method of determining the effectiveness of a pulmonary therapy, the method comprising: (a) measuring real-time respiratory data comprising: providing a device capable of connecting to a subject for obtaining the real-time respiratory data, the device comprising: a chamber comprising an interior, an inlet and outlet for air, wherein two or more first sensors are in fluid communication with the interior of the chamber, wherein the two or more first sensors are selected from a nitric oxide sensor, a nitrous oxide sensor, an oxygen sensor, an ozone sensor, a carbon dioxide sensor, a carbon monoxide sensor, a pressure sensor, a temperature sensor, an ammonia sensor, and a humidity sensor; one or more second sensors selected from an SpO₂ sensor or a PaCO₂ sensor; and a processor connected to each of the first and second sensors, and wherein the SIB system measures in real-time respiratory pressure and the inhalation and exhalation volumes of each breath of a subject; and calculating the real-time respiratory data with the processor; (b) administering a candidate drug to a first subset of the subjects, and a placebo to a second subset of the subjects; (c) generating the real-time respiratory data from the first and second subset of subjects; (d) calculating a difference between the real-time respiratory data in the first and second subset of subjects; and (c) if real-time respiratory data differs between the first and second subset of subjects then calculating an effectiveness of the pulmonary therapy.

As embodied and broadly described herein, an aspect of the present disclosure relates to a method of measuring respiratory function, the method comprising: connecting a device capable of connecting to a subject for obtaining the real-time respiratory data, the device comprising: a mask or chamber comprising an interior, an inlet and outlet for air, wherein two or more first sensors are in fluid communication with the interior of the chamber, wherein the two or more first sensors are selected from a nitric oxide sensor, a nitrous oxide sensor, an oxygen sensor, an ozone sensor, a carbon dioxide sensor, a carbon monoxide sensor, a pressure sensor, a temperature sensor, an ammonia sensor, and a humidity sensor; one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a subject; and calculating the real-time respiratory data with the processor to determine respiratory function.

### BRIEF DESCRIPTION OF THE DRAWINGS

For a more complete understanding of the features and advantages of the present invention, reference is now made to the detailed description of the invention along with the accompanying figures and in which:

FIG. 1 is a block diagram of the physical system including one possible layout of sensors mounted to a ‘body’ that connects to standard medical air tubing.

FIG. 2 is a block diagram of the electrical components and their interconnections, including external systems (e.g., a processor, a handheld device, or a computer) to extract and manipulate data, display graphs and save data.

FIG. 3 shows one example of the present invention in which the SIB system is attached between a standard positive airway pressure system.

FIG. 4 shows another example of the present invention in which the SIB system is attached between a standard positive airway pressure system.

FIGS. 5A to 5C are schematic diagrams of one example of electronics for use with the present invention.

FIG. 6 shows one example of a graphical user interface for use with the present invention.

FIG. 7 shows another example of a graphical user interface for use with the present invention.

FIG. 8A is a block diagram of the electrical components that comprise the core of the Data Logging Sensor Integration Block (DLSIB) system and a complement of sensors for pulmonary functioning evaluation of the DLSIB of the present invention.

FIG. 8B is a diagram of the physical components of an embodiment of the DLSIB system.

FIG. 9 shows a device in this application for human use.

FIG. 10 shows a device in this application for human use and the output from the system.

FIG. 11 shows another device in this application, in this case an equine/bovine model.

FIGS. 12A to 12F show drawings for various components of the equine/bovine embodiment of the DLSIB.

FIG. 13 shows a flowchart of a method of the disclosure.

FIG. 14 shows a flowchart of another method of the disclosure.

FIG. 15 is a graph that shows oxygen and carbon dioxide correlations.

FIGS. 16A and 16B are graphs that show nitric oxide readings of the horses.

FIGS. 17A and 17B are graphs that show maximum nitric oxide measures of the horses.

FIG. 18 is a graph that shows the difference between maximum and minimum nitric oxide.

FIG. 19 is a flowchart that illustrates a study timeline.

FIG. 20 is a graph that shows an Air Quality Index for Study Sites.

FIG. 21 is a mosaic plot of asthma phenotype by AQI.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention is a Data-Logging Sensor Integration Block (DLSIB) that can be inserted in-line between a patent's respiratory mask and any commercially available respirator or breathing device. This DLSIB records, logs, and analyzes a comprehensive dataset, and display this data to medical personnel in a manner that assists them with their diagnostic and therapeutic decisions and their medical evaluations in real time. The DLSIB can also be used as an educational module in a simulation scenario. The DLSIB can be used independently, that is, solely as a monitoring device. Thus, the DLSIB can be used as a sensor array in which a mask, that a patient wears for a brief or extended period of time, collects data pertaining to respiratory functioning, which monitors respiratory function. The monitoring device can be used to measure the patient's respiratory functioning when breathing room air naturally.

The system of the present invention displays and records, during each respiratory cycle of inhalation and exhalation, not only the tidal volume and the respiratory pressure, but also the total oxygen and carbon dioxide exchange with the blood in each breath. The system provides a direct measurement of, and the change over time in, the immediate change in blood oxygenation well before the PaO₂ indication responds. In the case of apparently healthy, deeply hypoxic patients, i.e., “silent hypoxia”, this system provides a blood oxygen exchange diagnostic that is not directly associated with tidal volume measurements, providing immediate feedback to the caregiver regarding the patient's significant pulmonary health indicators. This data can also be archived and is retrievable in an easy manner to answer voice-recognized queries rapidly and intuitively.

Thus, the present invention enables (1) obstruction detection by providing real-time feedback of respiratory pressure, inhalation volumes, and exhalation volumes for each breath, (2) real-time monitoring during procedures to improve dosing modification, and (3) the system has advanced display and reporting features that provide comprehensive and comparable patient data.

Another feature of the present invention is that it can be integrated into existing assistive respiratory devices to enable improved sensing capabilities not presently offered in most devices. The ability of the invention to enable detection of physiologic and physical obstructions that might be preventing optimal oxygen-carbon dioxide exchange at the alveolar level and to report these events in real-time could detect “silent hypoxia” in patients with normal tidal volume, a critical differentiator for this invention.

The most common triggers of asthma include allergies, air pollution, and other airborne irritants, respiratory infections, physical activity, weather conditions, strong emotions, and certain medications. These triggers can vary from person to person and even among different animals. It's known that nitric oxide synthases are abundant in the lungs, and nitric oxide (NO) is naturally present in exhaled air. It has been reported that the analysis of exhaled NO is increasingly recognized as a potential noninvasive test to assess the inflammatory aspect of asthma in patients. Elevated levels of exhaled NO have been observed in conjunction with cosinophilic inflammation and have shown correlation with other markers of inflammation in asthma. During asthma exacerbations, exhaled NO increases, while it decreases during recovery.

The current method for analyzing the total nitrites (NOx) in exhaled breath condensate (EBC) typically involves using a FENO analyzer. However, it's important to note that the diagnostic role of fractional exhaled NO (FENO) has limitations. Airway inflammation in asthma is heterogeneous, and increased FENO levels are not always associated with it (e.g., neutrophilic airway inflammation). For example, in patients already receiving inhaled steroids, the FENO test may yield false-negative results.

On the other hand, laminitis, also referred to as founder, is a devastating disease that poses a significant threat to a horse's well-being, potentially leading to severe consequences like career-ending conditions or even euthanasia. The main cause of laminitis is a decrease in blood supply to the foot, which results in intense pain and cell death. Stress is often a precursor to laminitis, depleting the body of essential chemicals known as “neurotransmitters.” Among these neurotransmitters, Nitric Oxide (NO), a gaseous substance, plays a vital role in relaxing the walls of blood vessels. It helps maintain open and unrestricted blood vessels, ensuring optimal blood supply to the affected area. NO stimulates the release of cyclic GMP, a compound that induces vasodilation and further enhances blood flow. However, when the Nitric Oxide-cyclic GMP pathway malfunctions, it has been linked to the development of various conditions like erectile dysfunction, myocardial ischemia, and asthma. Understanding and addressing this pathway's proper functioning are critical in managing and preventing the devastating effects of laminitis in horses. Active monitoring of Nitric Oxide (NO) levels and other respiratory parameters plays a crucial role in assessing a horse's health and determining the appropriate treatment for its recovery from laminitis. By closely monitoring NO levels, veterinarians and caretakers can gain valuable insights into the horse's condition, allowing for informed decision-making in its care. This approach aligns with the recent discussions held during the Havemeyer Workshop, where researchers and clinicians gathered to exchange insights and advancements on Equine Asthma. A key emphasis during the workshop was the need for accessible and standardized diagnostic tools, leading to the development of targeted treatment approaches.

Hence, it would be highly advantageous to develop analyzers capable of rapidly and reliably measuring various gas exchange parameters, such as oxygen (O₂), ozone (O₃), carbon dioxide (CO₂), carbon monoxide (CO), nitric oxide (NO), nitrous oxide (N₂O), ammonia, mass flow, temperature, pressure, and humidity. These analyzers should be portable and cost-effective, facilitating further clinical analysis to determine the causes and guide appropriate treatment.

The inventors disclose herein a Data-Logging Sensor Integration Block (DLSIB) unit that can read a variety of sensors, of either the analog output type, or “smart” sensors, and may be housed in different enclosures based on the end application. The system, comprising the main electronics, and the chosen sensors can be placed in a housing specifically meant for in-line readings between an individual and a CPAP or respirator unit, in a hand-held housing for clinical vitals, field deployment, or the consumer market, and finally in a housing designed for veterinarian use, e.g., livestock. The device can be used with any animal, including humans. Another use is in comparative medicine, for example, horse-human pulmonary studies. The invention records (at a user chosen sample rate) various parameters of gas exchange, including, but not limited to: oxygen (O₂), ozone (O₃), carbon dioxide (CO₂), carbon monoxide (CO), nitric oxide (NO), nitrous oxide (N₂O), ammonia (NH₃), mass flow, temperature, pressure, and humidity. Each recording is time and date stamped. The unit can make several recordings, limited only by set sample rate and available memory. Once data has been recorded in the non-volatile memory, it can be uploaded to a host computer for display, saving to local files, and analysis.

As used herein, the term “Data Logging Integration Sensor Block” or “DLSIB” refers to an electronic system that measures, and records to non-volatile memory one or more of the following: the respiratory pressure, the inhalation and exhalation volumes on each breath (bi-directional mass-flow), the O₂, O₃, CO₂, NH₃, and/or NO level(s) of each breath, and/or the temperature of the flowing gasses. Additional measurements can be added to the DLSIB system, such as SpO₂, heart rate variables such as heart beats per minute, changes in heart rate over time, body temperature, etc. The device can also display, in real-time these measurements if connected to a computer. A Windows-based software suite has been written and can be downloaded or display data from the DLSIB.

As used herein, the phrase “Sensor Integration Block (SIB)” refers to a system that measures, in real-time, the respiratory pressure, the inhalation and exhalation volumes on each breath, the variation in the partial pressure of O₂ and CO₂ during the breath, and the integrated quantity of oxygen added to the blood, and the integrated quantity of CO₂ removed from the blood, during each breath. This information may be used to infer physiologic and/or physical obstructions that prevent optimal oxygen-carbon dioxide exchange at the alveolar level of gas exchange, thus enhancing and accelerating diagnostic and therapeutic intervention for pulmonary hygiene such as suctioning, incentive spirometry, percussion, vibration, postural drainage, and/or breathing exercises.

The DLSIB provides various reporting functions to assure that the current health, and immediate health history, are available to caregivers on various time scales. These reports are described in more detail herein below.

In real-time, during patient procedures and/or the dosing of patient medication, the caregiver will be able to see the immediate level, and change of level over the last few breaths, of the level of gas exchange to the blood during each respiratory cycle. This will also include tidal volume variations and respiratory pressure, and an indicator of how these quantities are trending. This information is useful for making changes in the patient's care and medications in real time, before slower indicators, such as PaO₂, respond.

Over the course of minutes, the general trend in the patient's respiratory efficiency can be displayed in an intuitive, easily understood plot. The caregiver can set alert levels and audible trend alarms to draw attention to sudden changes in these patient trends. This short-term data can be easily compared to the respiratory history of the patient over a much wider time interval using an intuitive interface.

On the change of personnel shifts, the DLSIB system can provide a comprehensive summary of the patient's respiratory history, along with other vital data and caregiver notes, to assure proper continuity of care and emphasis on significant ‘deltas’ in parameters. These reports can be tailored to the severity of the patient's condition, ranging from sleep apnea monitoring to intubated respirator-supported patients, high altitude pulmonary effects in the field, as well as effects in space conditions or during transport to space.

In another aspect, the system can continuously assimilate this DLSIB system data, and provide a regressive analysis of the patient's performance during earlier time intervals. An intuitive interface provides the caregiver with detailed histories and correlations with other vital signs over a period that is specified by the caregiver. The system can compare to other cohort patient populations, and provide an easily understood comparison to the patient's respiratory performance to others within the patient's cohort across the general population. Data is provided in a fully disaggregated manner to an artificially intelligent (AI) system that is capable of detecting more complex health indicators through advanced pattern recognition in large, multi-patient data sets for future display and analysis of the patient's condition by the health care professional.

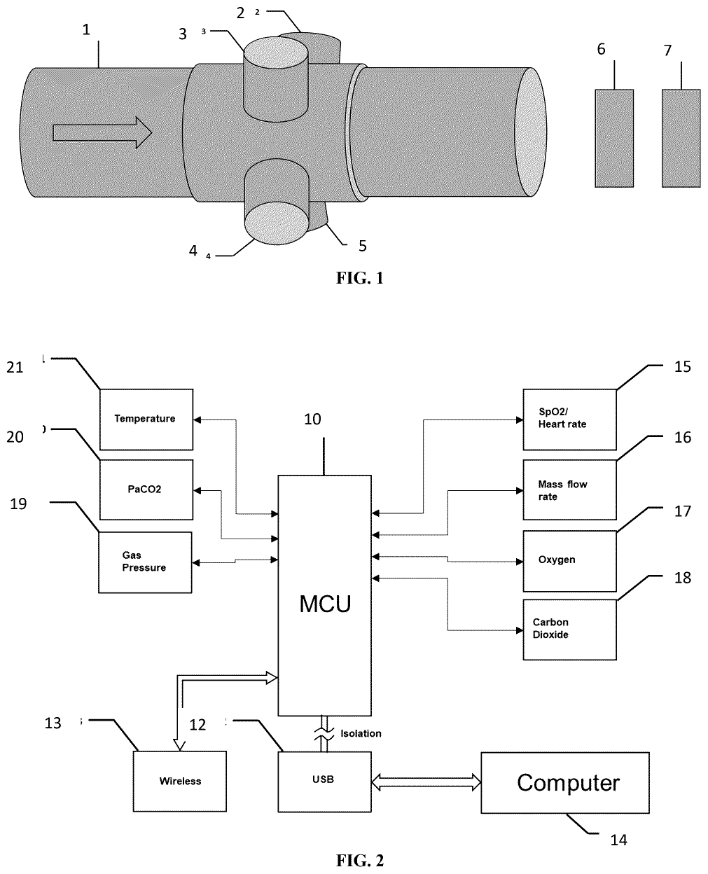

FIG. 1 is a block diagram of the physical SIB system including one possible layout of sensors mounted to a ‘body’ that connects to standard medical air tubing. Air from the individual being monitored enters from external tubing that connects to a bi-directional mass flow sensor (1). Air then enters the sensor system's main body where it expands to connect with an oxygen sensor (2), a carbon dioxide sensor (3), a pressure sensor (4), and a temperature sensor (5). Simultaneously, an SpO₂ sensor (6) and a PaCO₂ sensor (7) are monitored that are either attached to the individual's ear or finger to monitor heart rate, oxygen saturation and PaCO₂ connected to the same host device (e.g., a processor, a handheld device, or a computer), or to different devices that are interconnected, which communications can be wired or wireless.

FIG. 2 is a block diagram of the electrical components and their interconnections, including external systems (e.g., a processor, a handheld device, or a computer) to extract and manipulate data, display graphs and save data. A generic microcontroller (10) communicates with the external processing and display system (e.g., a computer) (14) via any standard wired communication protocol such as USB (12) or a RS-485, RS-232, etc., or via any standard wireless data protocol such as Bluetooth, Wi-Fi, Zigbee, etc. The microcontroller (10) collects sensor data in analog form, or digitized form, from: an SpO₂/heart rate sensor (15), a mass flow sensor (16), an oxygen sensor (17), a carbon dioxide sensor (18), a pressure sensor (19), a PaCO₂ sensor (20), and a temperature sensor (21). Data sampling time can be periodic or arbitrary within the bounds of the microcontroller(s) (10) and the attached sensor devices (15, 16, 17, 18, 19, 20, 21) frequency limitations, which allows flexibility in choosing the desired temporal resolution for monitoring the various parameters during each breathing cycle.

An external processing and display system contains advanced algorithms that computes derived parameters from the fundamental quantities measured by the sensors. A Graphical User Interface (GUI) displays desired data in the form of graphs as well as the real-time collected data.

Data is saved on the processing and display unit in a format chosen by the user such as the Comma Separated Value (CSV) file format.

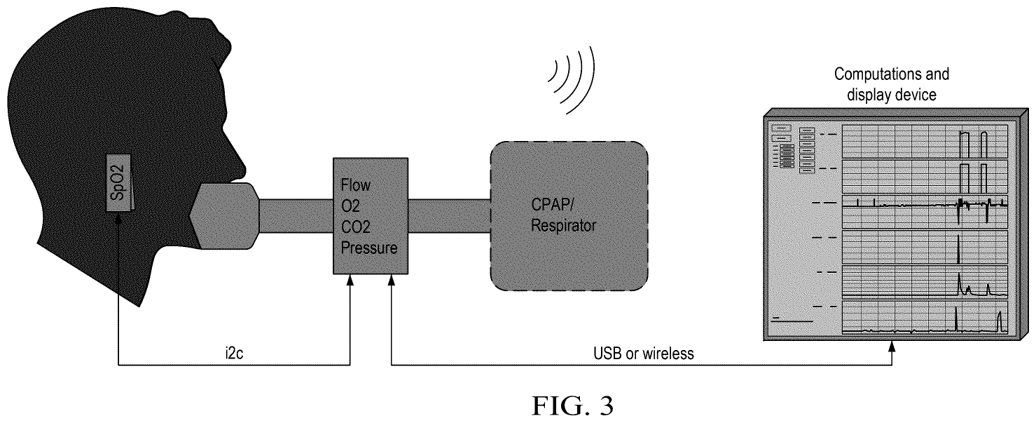

FIG. 3 shows one version of the present invention in which the SIB system is attached between standard positive airway pressure systems such as a continuous positive airway pressure (CPAP), an automatic positive airway pressure (APAP), a Bilevel, or variable positive airway pressure (VPAP), a respirator, or equivalent, and the patient mask. In some examples, the SIB system of the present invention can be integrated into the mask, the tubing, or even at the air pump device. In this example, the SpO₂ sensor is connected to, e.g., an car of the patient. These connections can be wired and/or wireless.

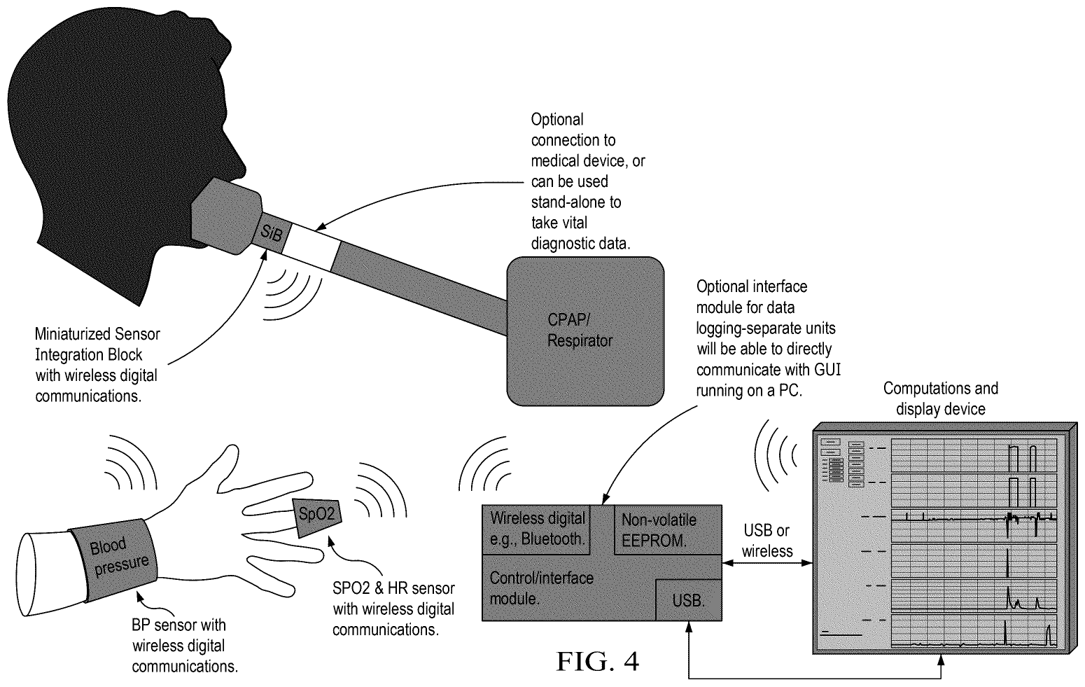

Alternatively, as shown in FIG. 4, it is possible to use separate “autonomous” units for blood pressure (BP), SpO₂ and the gas measurements allow for easily acquiring data from sensors with highly disparate sample rates as well. The incorporation of all these sensors means that nearly every important parameter of a person's present state of health is collected (and recorded) by diagnostic equipment, with the only addition to the “standard” apparatus currently being the addition of a small mask the patient wears. All the parameters are collected in one place, with accurate timestamps. From the data itself, the attending clinician can see at a glance the most important vitals, and more in-depth algorithms can calculate derived parameters. The SIB can be used independently, that is, solely as a monitoring device. Thus, the SIB can be used as a sensor array in which a mask, that a patient wears for a brief or extended period of time, collects data pertaining to respiratory functioning, which monitors respiratory function. The monitoring device can be used to measure the patient's respiratory functioning when breathing room air naturally.

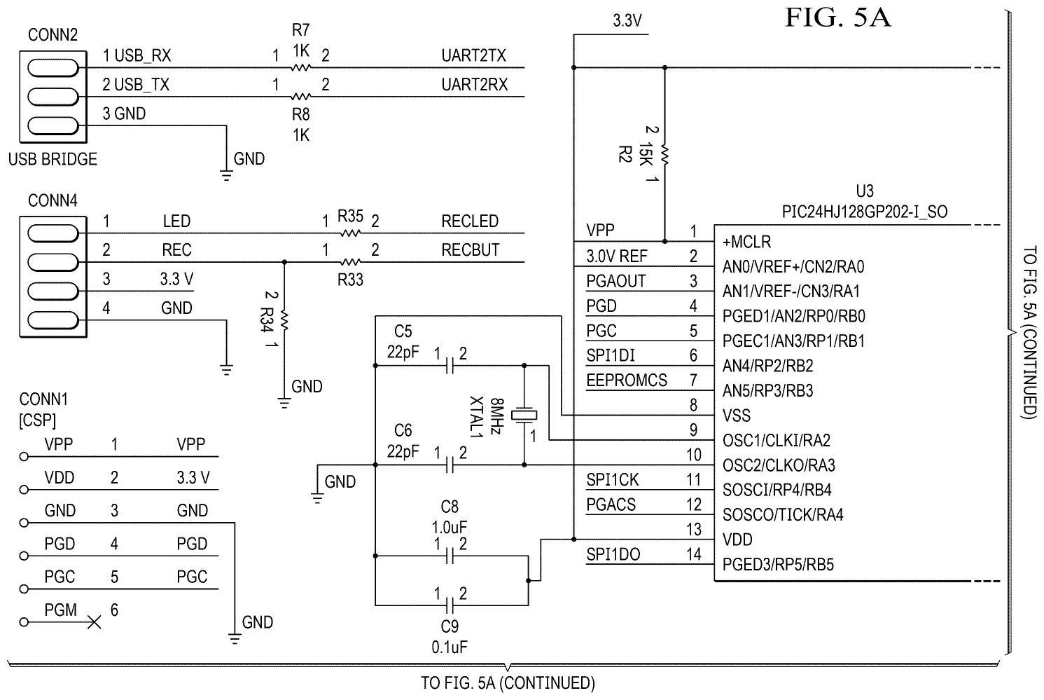

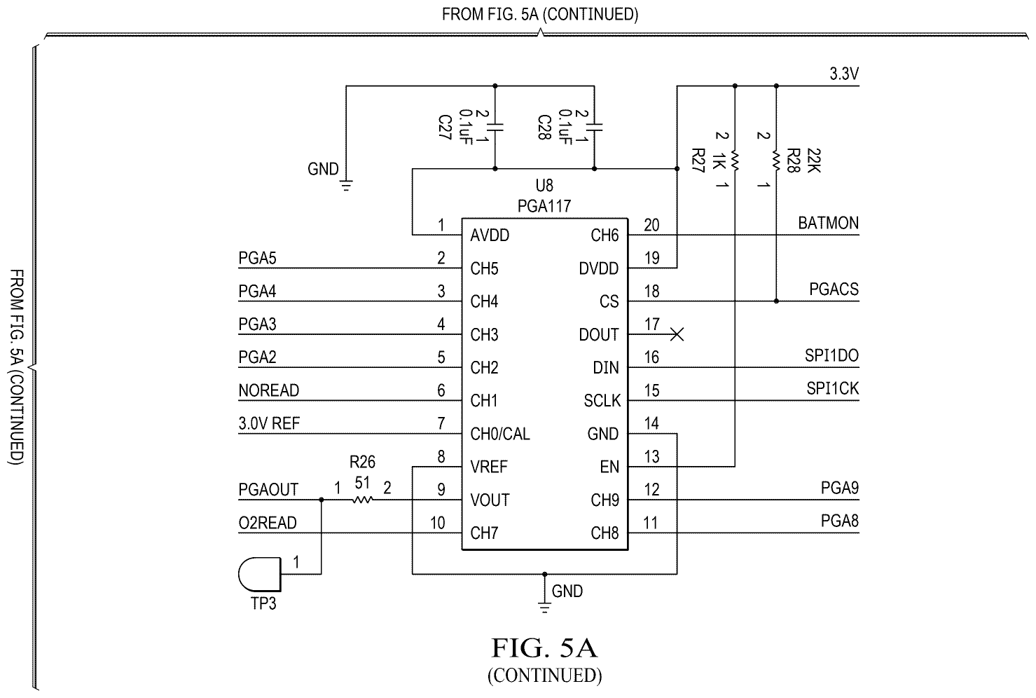

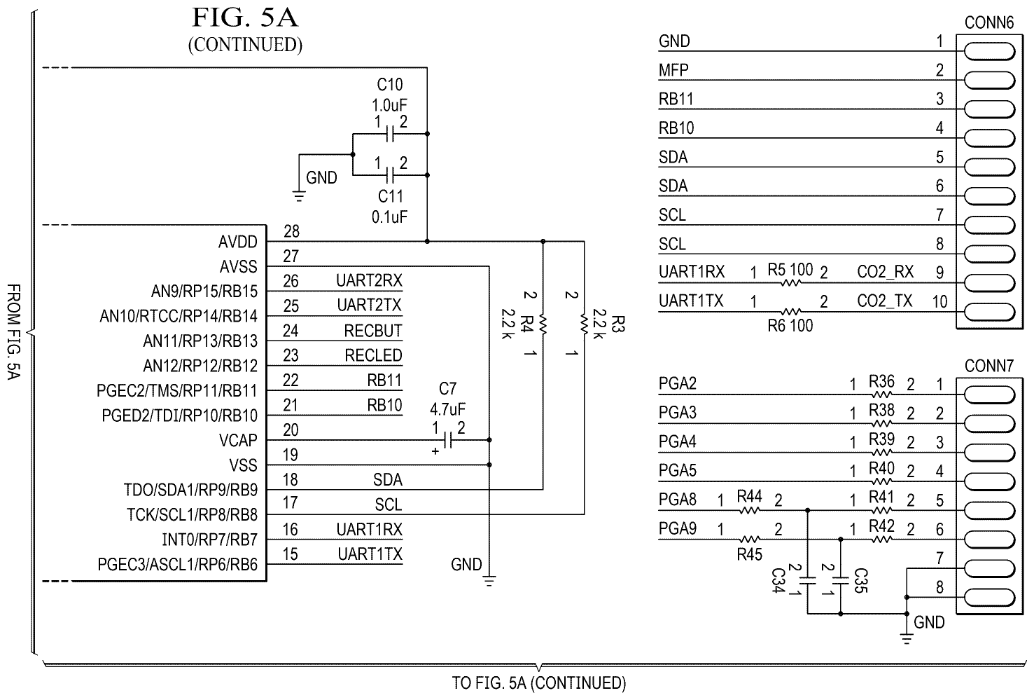

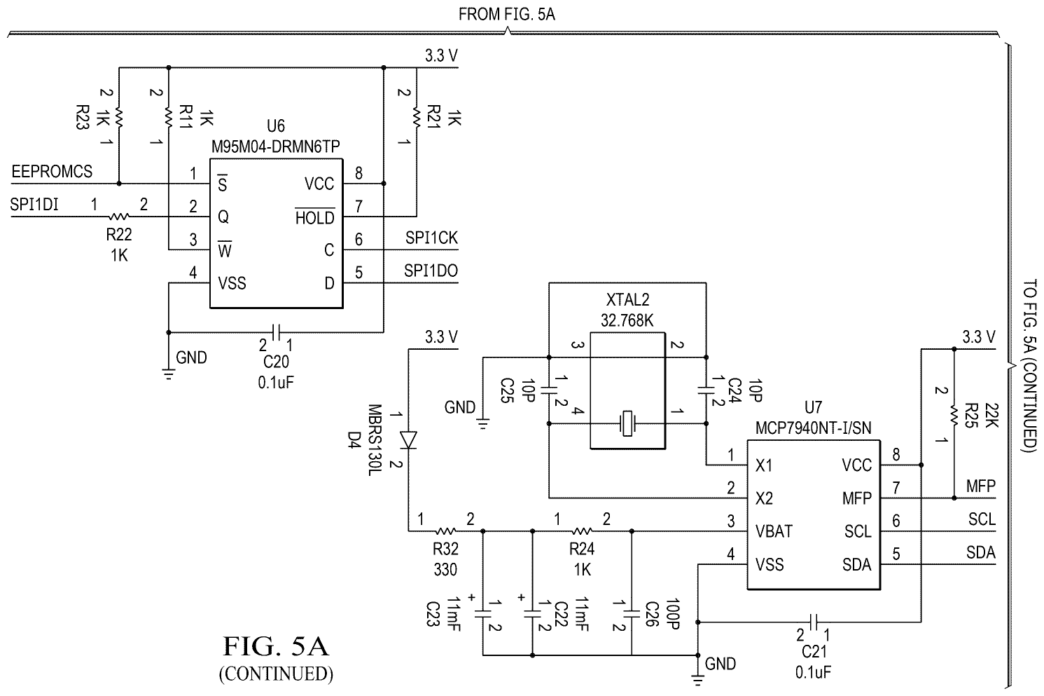

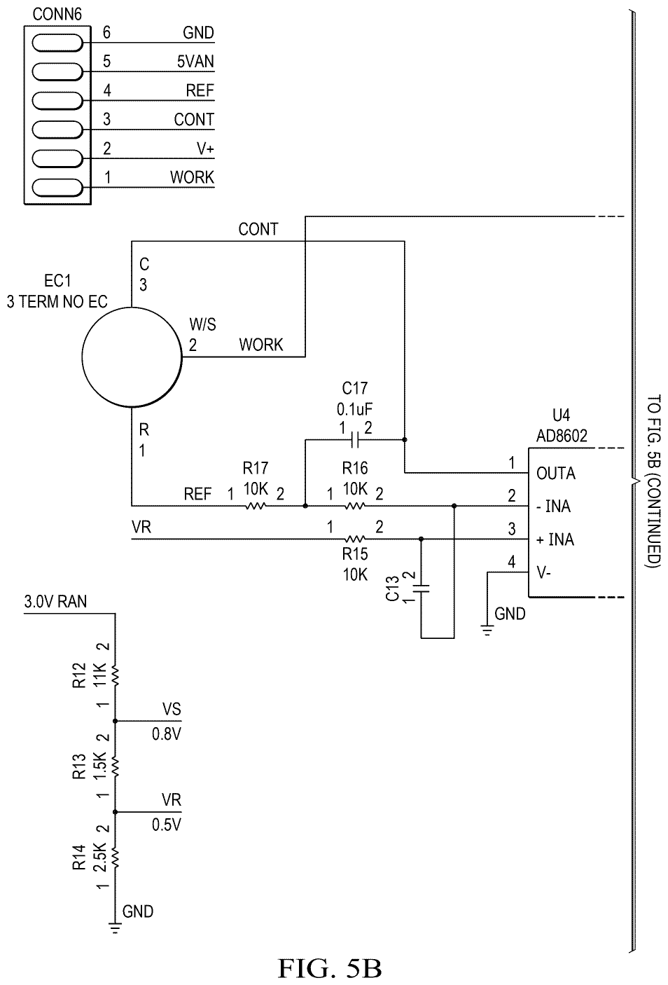

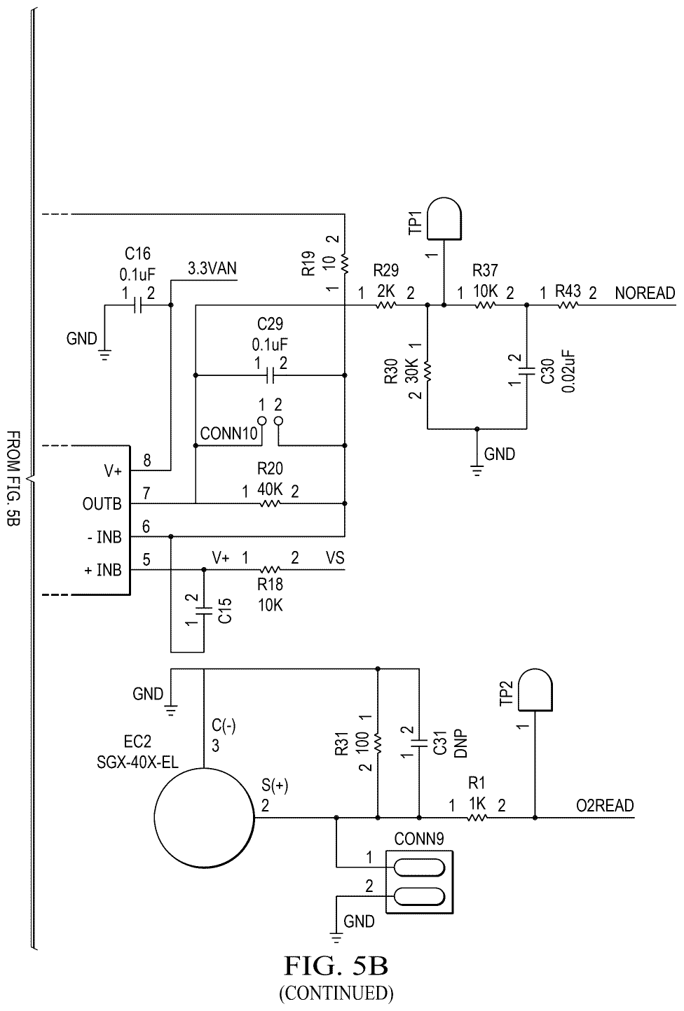

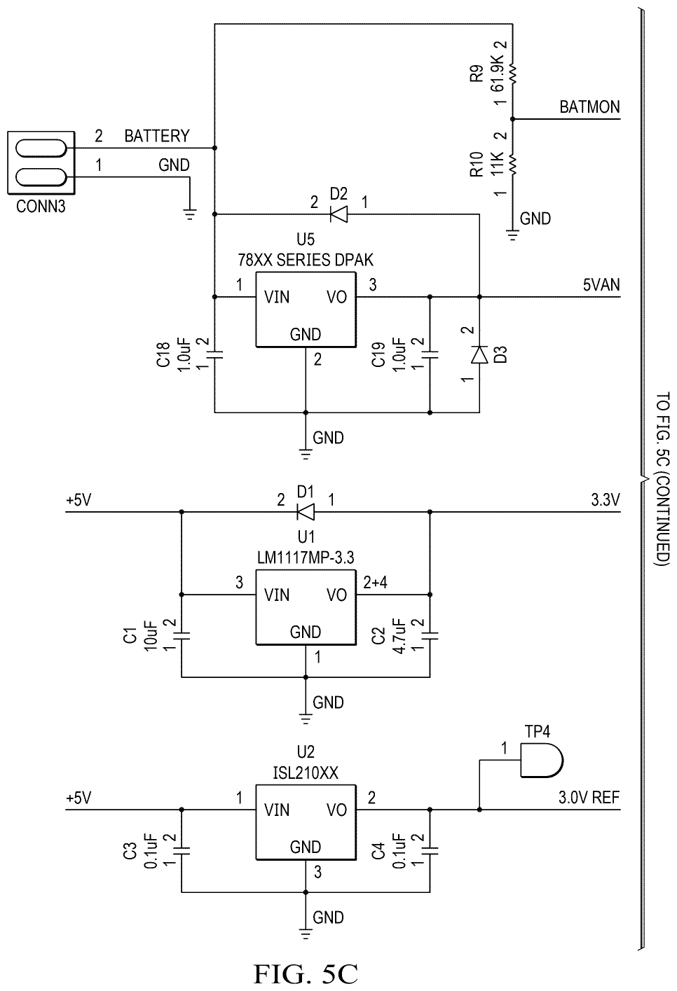

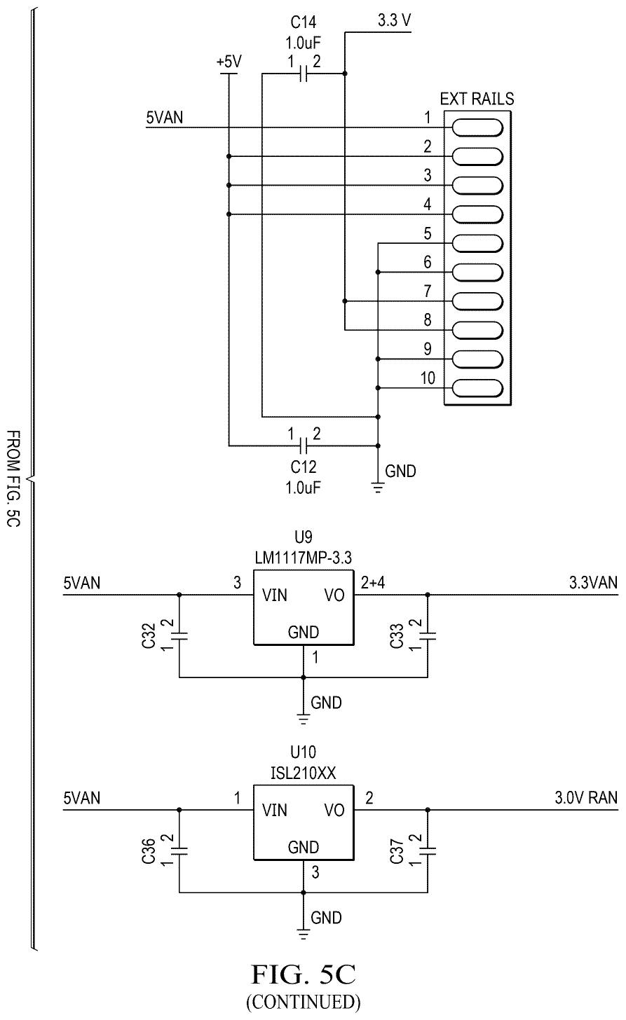

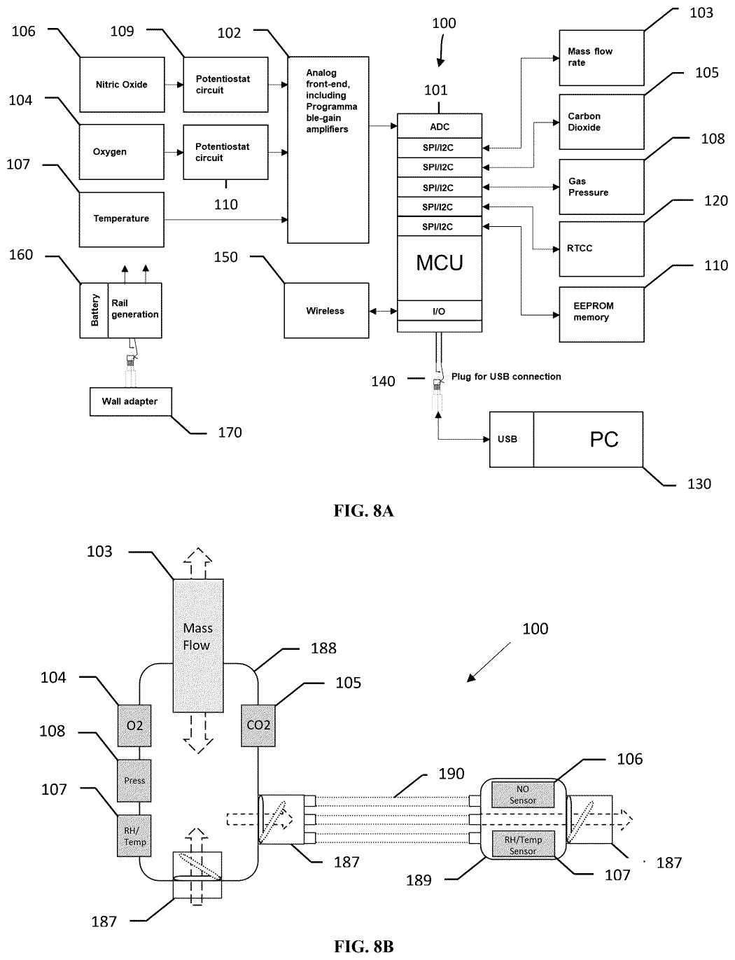

FIGS. 5A to 5C are schematic diagrams of one example of electronics for use with the present invention. This schematic matches the inputs shown in FIGS. 8A and 8B.

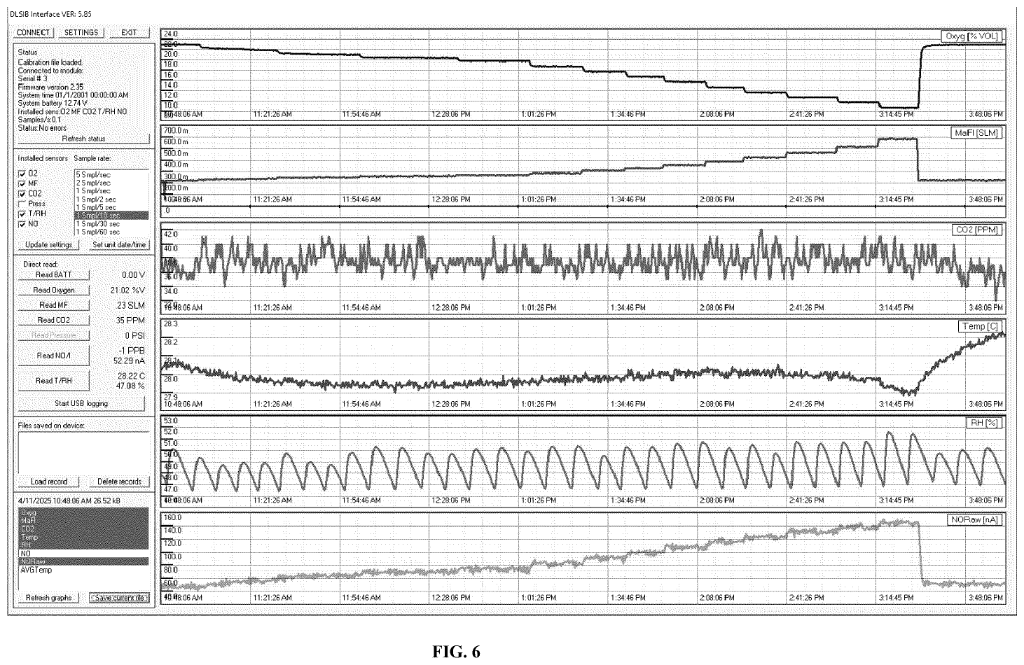

FIG. 6 shows one example of a graphical user interface for use with the present invention. This example shows the various data (an SpO₂/heart rate sensor (15), a mass flow sensor (16), an oxygen sensor (17), a carbon dioxide sensor (18), a pressure sensor (19), a PaCO₂ sensor (20), and a temperature sensor (21)) in separate graphs that allows for the clinician to monitor each individual data stream separately.

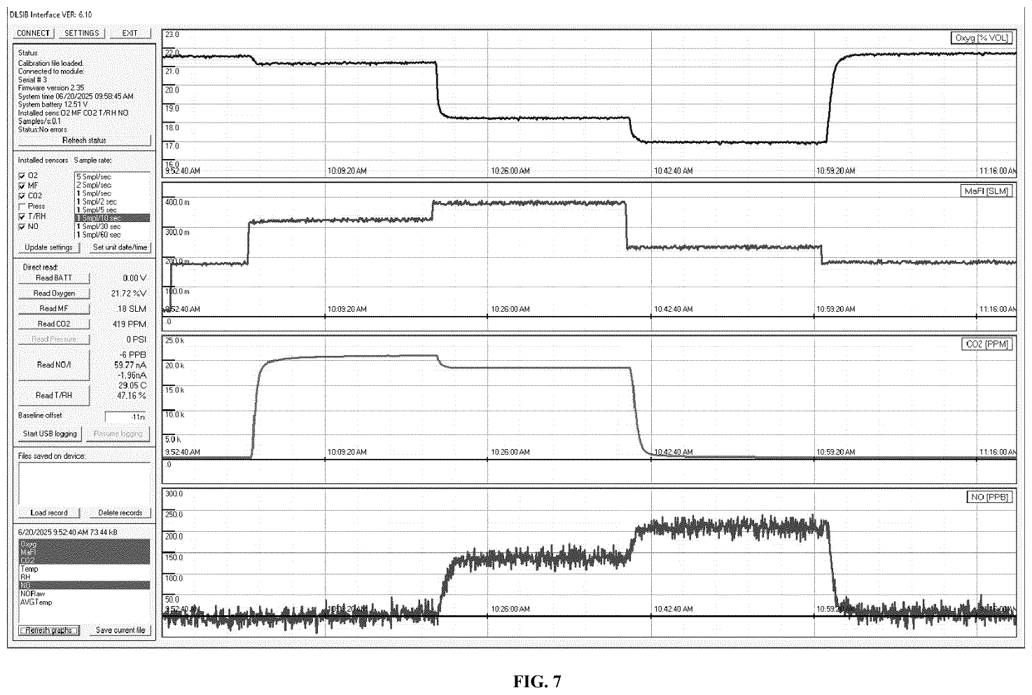

FIG. 7 shows another example of a graphical user interface for use with the present invention. In this example, the various data (an SpO₂/heart rate sensor (15), a mass flow sensor (16), an oxygen sensor (17), a carbon dioxide sensor (18), a pressure sensor (19), a PaCO₂ sensor (20), and a temperature sensor (21)) on the same graph that allows for the clinician to monitor the aggregated data streams.

FIG. 8A is a block diagram of the electrical components that comprise the core of the DLSIB system 100, along with what is considered a minimum complement of sensors for pulmonary functioning evaluation. A generic microcontroller (101) collects sensor data either in analog form from those sensors that produce a voltage or current output via the analog front end (102), or in digitized form from “smart” sensors using various digital communication protocols.

This example diagram shows a mass flow sensor (103), an oxygen sensor (104), a carbon dioxide sensor (105), a nitric oxide sensor (106), a temperature sensor and relative humidity sensor (107), and a pressure sensor (108) connected to the core system. In addition, additional sensors can be included such as an O₃ and/or NH₃ sensor (not depicted). The system has built-in analog processing, including specialized Electrochemical read out subsystems such as potentiostat circuits (109-110) to read the raw output of certain Electrochemical Cells (ECs). Data sampling time is arbitrary and settable from a GUI (when the unit is connected to a PC) within the bounds of the microcontrollers and the attached sensor devices frequency limitations, which allows flexibility in choosing the desired temporal resolution for monitoring the various parameters during a breathing cycle. The collected data is saved in a file on the Electrically Erasable Programmable Read Only Memory (EEPROM) (110), and each file is stamped with the time and date of recording acquired from the Real Time Clock Calendar (RTCC) (120). The system communicates with an external processing and display system, e.g., a PC (130) via a detachable plug (140) through USB, or via any standard wireless data protocol such as Bluetooth, Wi-Fi, LoraWAN RF standard, etc. (150). A Graphical User Interface (GUI) displays either downloaded files, or can display data in real-time on a monitor. Data can also be saved on the local processing and display unit in a format chosen by the user such as the Comma Separated Value (CSV) file format. The unit can be battery operated (160), or powered via a wall adapter (170).

FIG. 8B is a diagram of the physical components of an embodiment of the DLSIB system 100. The DLSIB system 100 includes a mass flow sensor 103, an oxygen sensor 104, a carbon dioxide sensor 105, a nitric oxide sensor 106, a temperature and relative humidity sensor 107, a pressure sensor 108, one-way check valves 187, a main housing 188, a nitric oxide housing 189, and moisture exchange tubing 190 (e.g., NAFION® tubing). The nitric oxide sensor 106 requires readings from a temperature and relative humidity sensor 107 inside the same housing. Some embodiments have a separate temperature and relative humidity sensor 107 in the direct stream of exhaled breath.

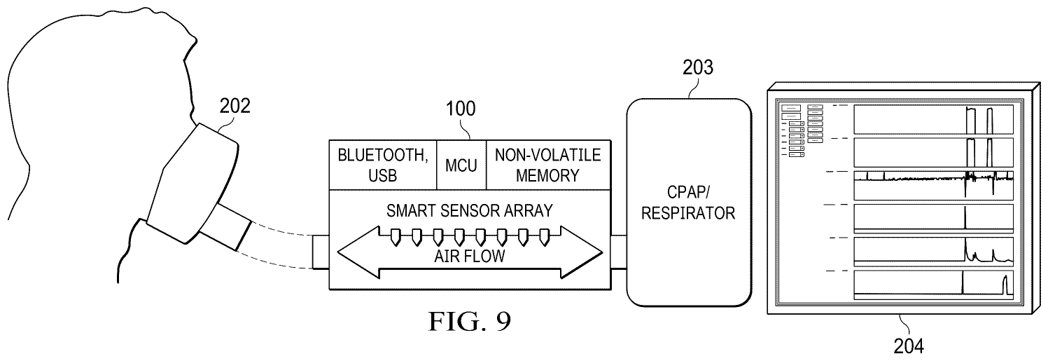

One application of the DLSIB is for pulmonary/respiratory monitoring of patients using breathing equipment such as CPAP machines or Respirators. FIG. 9 shows the device in this application. The DLSIB (100) is placed in-line between the patient's mask (202) and the pulmonary apparatus (203). Data can either be logged to memory or displayed real-time on a monitor (204) for attending physicians or nursing staff. Much like other medical monitoring systems, alarms can easily be incorporated into the software. “Patient” refers to a human subject or an animal subject, and the term “subject” is used herein as an equivalent of “patient.”

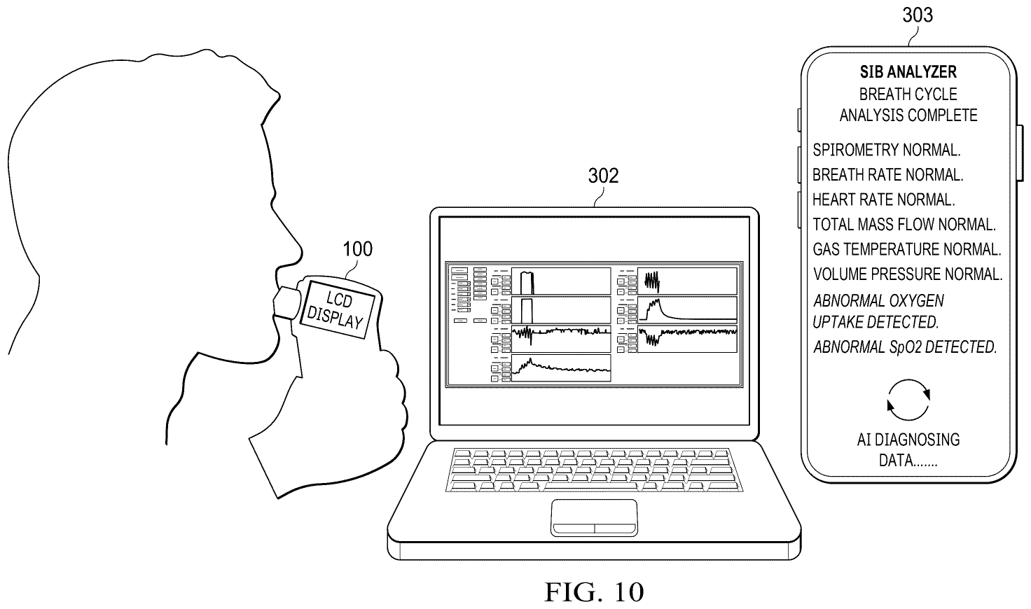

Another application of the present invention is a handheld version. This aspect of the device allows extensive breathing gas-exchange data to be taken at any time in any location. Several examples would be taking data during normal patient clinical vitals, emergency medicine situations, e.g., ambulance, and air-borne critical care. The device is also suitable for the consumer market much like portable SPO₂ meters. FIG. 10 shows the invention in this application, the core and sensors are housed in a handheld device (100), data can then either be uploaded to a PC (302), or wirelessly transferred to a smartphone app (303). Outputs from the handheld device (100) can include: spirometry, breath rate, heart rate, total mass flow, gas temperature, volume pressure, oxygen uptake, SpO₂, etc., which can also be output as graphs.

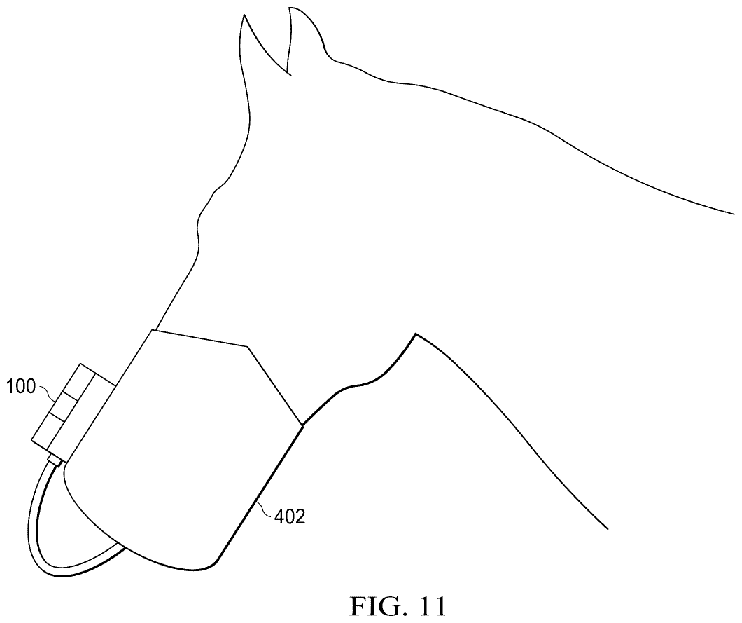

Yet another manifestation of the invention is for use in the veterinarian fields. FIG. 11 shows this application. The DLSIB (100) would be connected in some manner to a mask attached to the animal's mouth and nose (402). The portable and battery operated nature of the DLSIB then allows animals to roam free in their natural environment while gas exchange, temperature and pressure are recorded.

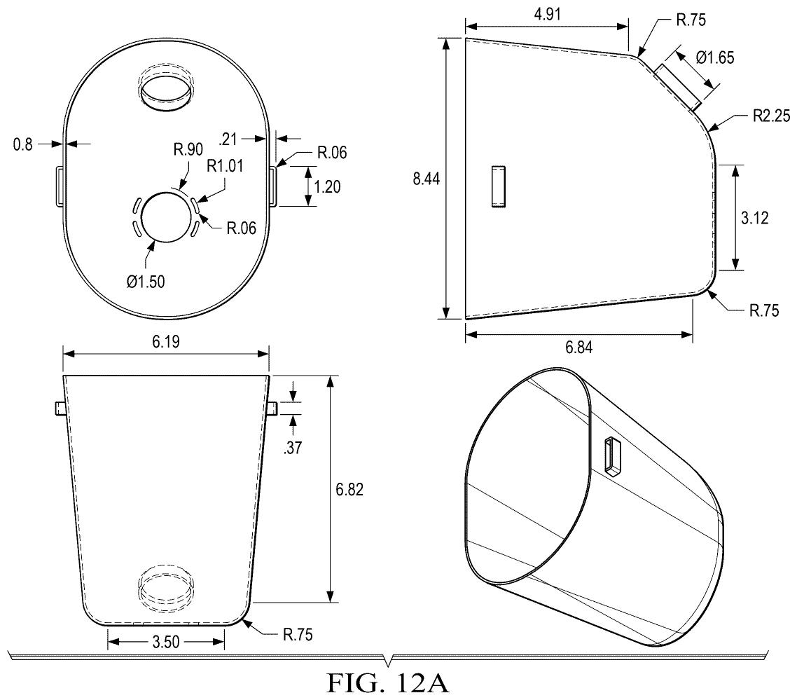

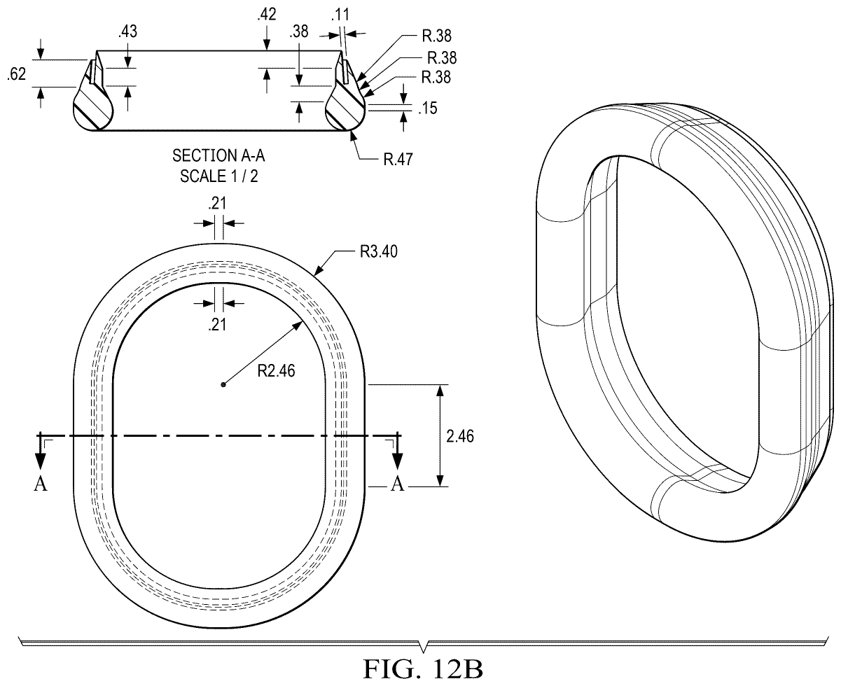

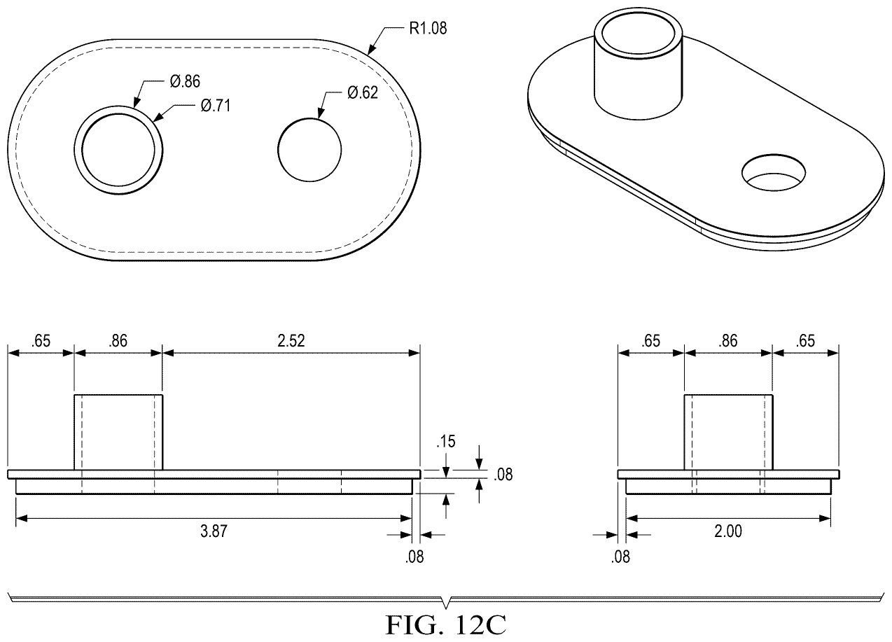

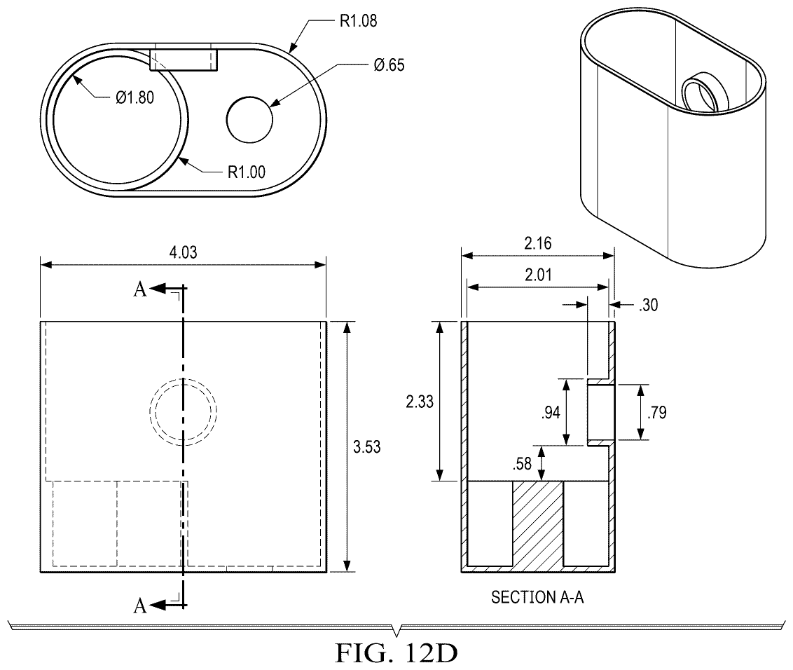

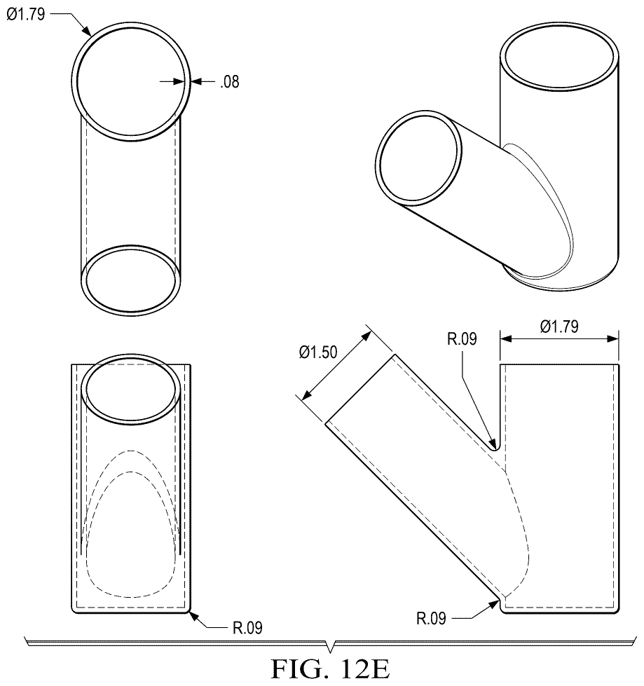

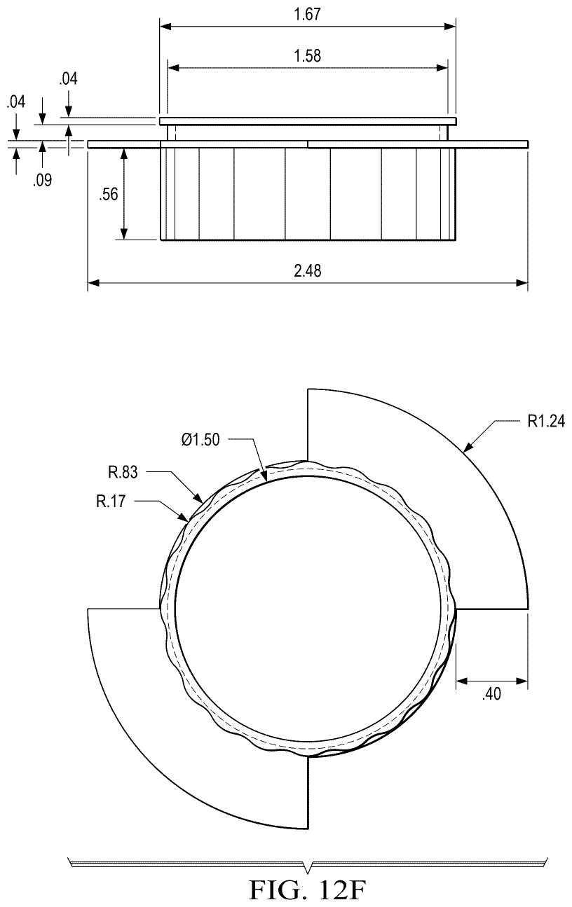

FIGS. 12A to 12F show drawings for various components of the equine/bovine embodiment of the DLSIB (100).

The DLSIB system 100 was tested. The cost of 3D printing the body part was very reasonable, making it an affordable option. The well-balanced primary body part is constructed using, e.g., flexible PLA with a hardness rating of 89A, which is highly comparable to other products available in the market and has the added advantage of being cost-effective. While all of the measurements possible with the DLSIB can be made via separate medical tests, e.g., CO₂ (capnography), mass flow (spirometry), O₂, O₃, NH₃, etc., the DLSIB measures all of these simultaneously to achieve a synergistic view of all the parameters. This synergistic view of all the parameters shows the interactions between them and is a far more powerful diagnostic tool.

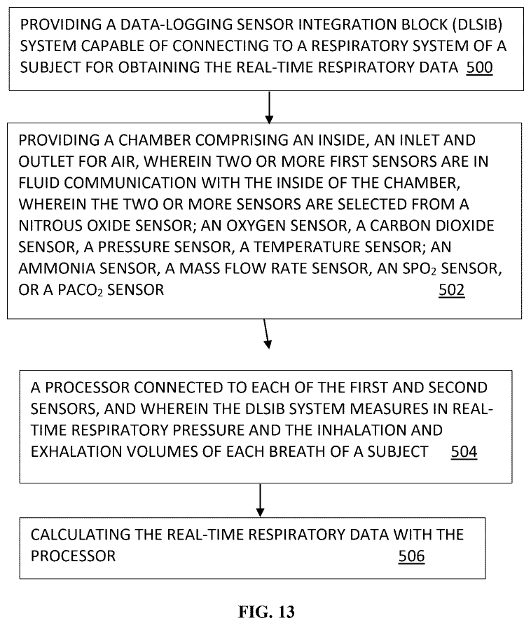

FIG. 13 shows a flowchart of a method of the disclosure. The method begins at step 500, which includes providing a Data-Logging Sensor Integration Block (DLSIB) system capable of connecting to a respiratory system of a subject for obtaining the real-time respiratory data. Next, in step 502, providing a chamber comprising an interior or inside, an inlet and outlet for air, wherein two or more first sensors are in fluid communication with the interior or inside of the chamber, wherein the two or more sensors are selected from a nitric oxide sensor, a nitrous oxide sensor an oxygen sensor, an ozone sensor, a carbon dioxide sensor, a carbon monoxide sensor, a pressure sensor, a temperature sensor, a humidity sensor a mass flow rate sensor, an SpO₂ sensor, or a PaCO₂ sensor. In step 504, providing a processor connected to each of the first and second sensors, and wherein the DLSIB system measures in real-time respiratory pressure and the inhalation and exhalation volumes of each breath of a subject. Finally, in step 506, calculating the real-time respiratory data with the processor.

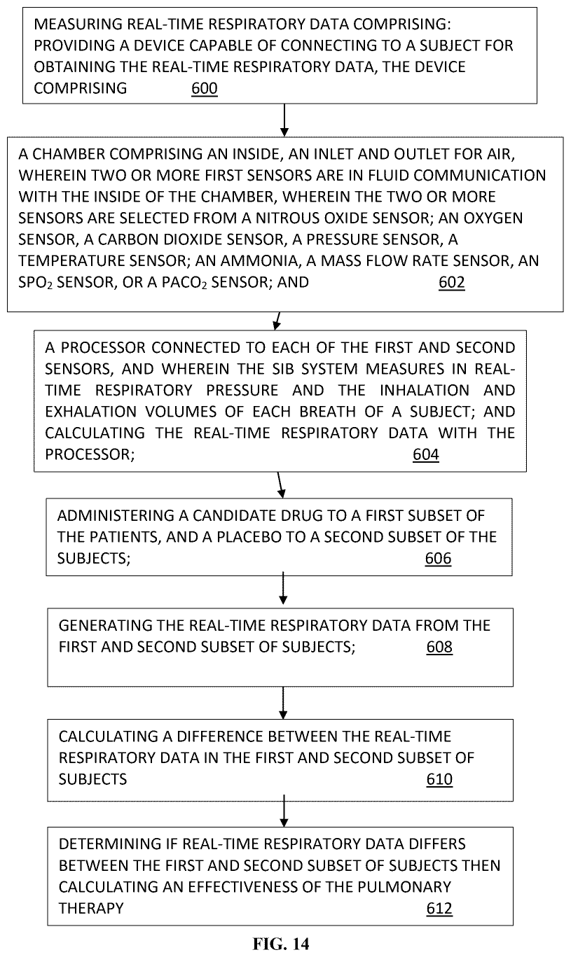

FIG. 14 shows a flowchart of another method of the disclosure. The method begins at step 600 by measuring real-time respiratory data comprising: providing a device capable of connecting to a subject for obtaining the real-time respiratory data. The device in step 602 includes a chamber comprising an interior or inside, an inlet and outlet for air, wherein two or more first sensors are in fluid communication with the interior or inside of the chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, a mass flow rate sensor, an SpO₂ sensor, or a PaCO₂ sensor, and in step 604 a processor connected to each of the first and second sensors, and wherein the DLSIB system measures in real-time respiratory pressure and the inhalation and exhalation volumes of each breath of a subject; and calculating the real-time respiratory data with the processor. Next, in step 606, administering a candidate drug to a first subset of the subjects, and a placebo to a second subset of the subjects. In step 608, generating the real-time respiratory data from the first and second subset of subjects. In step 610, calculating a difference between the real-time respiratory data in the first and second subset of subjects. Finally, in step 612, determining if real-time respiratory data differs between the first and second subset of subjects then calculating an effectiveness of the pulmonary therapy.

The nitric oxide sensor can be configured to take serial measurements of nitric oxide exhaled by an animal. In certain examples, the animal is a large mammal such as an equine, bovine, or other domesticated or non-domesticated mammal, and can also be used with humans. Traditionally, it is difficult to collect a sufficient nitric oxide sample from a mammal, such as an equine or bovine, to be of use for diagnosis of asthma or other respiratory diseases or conditions. It should be appreciated that the system disclosed herein can be used for the detection of other conditions including, but not limited to, pneumonia and other such respiratory diseases or conditions, such as respiratory distress, chronic obstructive pulmonary disease, emphysema, bronchitis, elevation hypoxia, or inhalation of soot from fires, vog from volcanic eruptions, dust from burn pits, and dust inside mining shafts. The disclosed embodiments make use of a collection muzzle, which is fitted snugly around the equine or bovine's nose, to allow a sufficient sample to be collected in a collection chamber from the horse's exhalation in a sufficient concentration to be measured.

In some embodiments, the nitric oxide (NO) sensor is an electrochemical (EC) sensor. The NO EC sensor must be compensated for temperature and absolute humidity values to obtain viable readings in the trace gas regime desired. This is accomplished by subjecting the sensor to known, controlled steps of temperature and absolute humidity to obtain mathematical correction factors for both the EC NO sensor's baseline current, and sensitivity (excess above baseline) currents. Also, humidity transients severely affect the baseline current, and cannot be compensated for in this manner. To mitigate humidity transients, a system utilizing moisture-exchange tubing (e.g., NAFION® tubing) has been devised, whereby the gas sample of interest is passed through parallel lengths of this tubing to equilibrate relative humidity (RH) with ambient humidity levels.

It should be appreciated that most of a horse's breath is captured during a nominally 3-second exhalation. In some embodiments, this can be completed pre-exercise and post-exercise so that measurements can be compared. An advantage of the current embodiments is that the horse need not hyperventilate to collect a sufficient sample.

The nitric oxide sensor can then provide quantifiable measurements of nitric oxide through continuous readings. In certain embodiments, this could comprise readings taken at, e.g., 0.1, 0.2, 0.5, 0.75, 1, 2, 3, 4, 5, 6, 7, 8, 9, 10, 15, 20, 25, 30, 45, or 60 minute intervals, but other sample timing is also possible.

The nitric oxide reading from the nitric oxide sensor can be provided to a processing system for analysis and outputting measurement information. If readings from the nitric oxide sensor suggest an animal has elevated nitric oxide, as measured in the collected exhalation form the animal, then the animal is likely suffering from non-infectious pulmonary inflammation or a viral infection. If the measurements are normal, then the animal is likely not-suffering from non-infectious pulmonary inflammation or a viral infection. If the measurements are high, treatment is warranted which would usually coincide with nebulization (albuterol & budesonide) and/or straight saline.

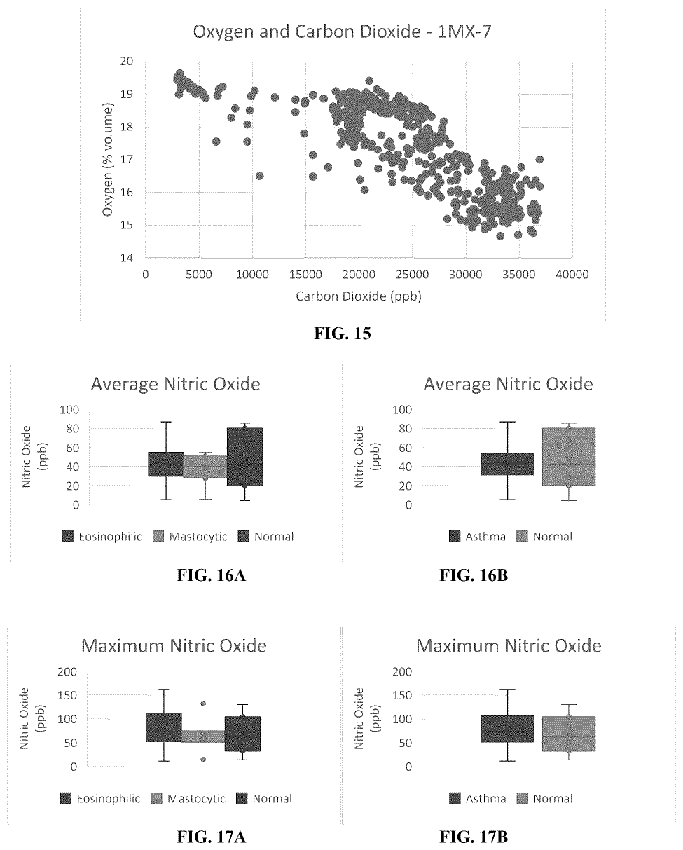

FIG. 15 is a graph that shows oxygen and carbon dioxide correlations. Mask data was collected from 20 horses at Mariposa Station. Real-time oxygen (measured as % volume) and carbon dioxide (measured as ppb) were recorded for all horses. Strong correlations that were statistically significant were found between oxygen and carbon dioxide measures in all horses.

Correlations ranged from −0.4905 to −0.8727, with a mean correlation of −0.679. All individual correlations had a p value <0.0001. A graph from a single horse is provided as an example.

These data indicated that there was a strong inverse relationship between measured exhaled oxygen and carbon dioxide. This is reflective of the known physiologic relationship between these two gases in exhaled breathe. This is the first known reporting of real-time exhaled breath measures in the awake horse.

FIGS. 16A and 16B are graphs that show nitric oxide readings of the horses. FIGS. 16A and 16B show the corrected nitric oxide readings of the horses evaluated at Mariposa Station. Five horses were diagnosed with both mastocytic and cosinophilic asthma, 14 horses were diagnosed with cosinophilic asthma only, 2 horses were diagnosed with mastocytic asthma only, and 1 horse was diagnosed with mastocytic and neutrophilic asthma. Seven horses did not have asthma. The 1 horse with mastocytic and neutrophilic asthma was grouped with the mastocytic asthma only horses where applicable. Asthma diagnoses were based on bronchoalveolar lavage fluid cytology.

Real-time nitric oxide measures were reported as both positive and negative values. Negative values were censured from the dataset, and only positive values were included in the analysis. The average, maximum, and difference between maximum and minimum nitric oxide values were reported for each horse. The average nitric oxide measures of the horses without asthma was so variable as to not detect a difference in comparison to horses with asthma.

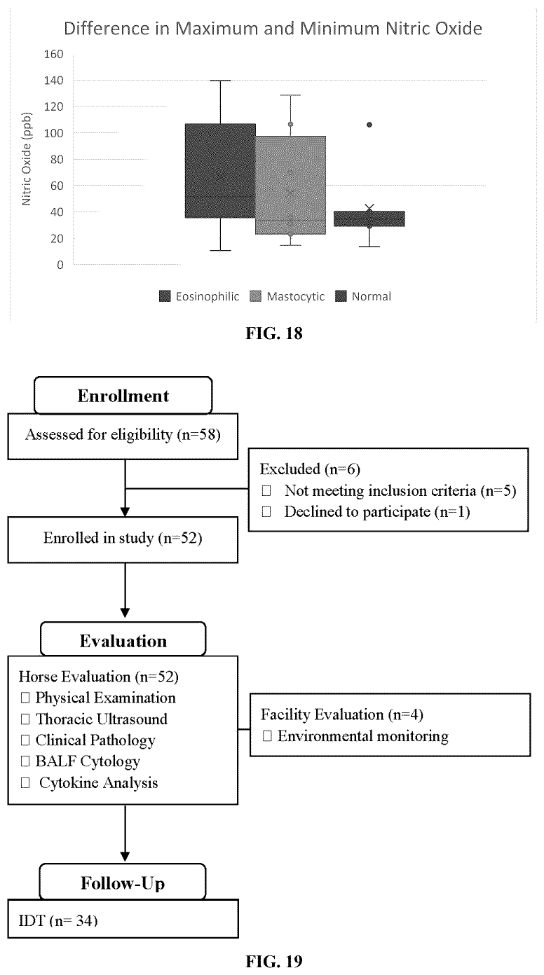

FIGS. 17A and 17B show maximum nitric oxide measures of the horses. The maximum nitric oxide measures of the horses without asthma was so variable as to not detect a difference in comparison to horses with asthma. FIG. 18 is a graph that shows the difference between maximum and minimum nitric oxide.

Allergic asthma in horses has been poorly characterized to date. Various studies have investigated the role of environmental allergens in horses with respiratory inflammation.^{1,2} Genetic predisposition has been evaluated, and control of exposure to potential inflammatory antigens remains a common therapeutic recommendation; albeit one with significant challenges to implement for the owner.^{1,2} Allergy testing has been evaluated in horses with concurrent atopic clinical signs, including dermatitis or seasonal clinical signs.^{3,4} Environmental factors ranging from known allergens (pollens, weeds, grasses) to airborne particulates have been demonstrated to exacerbate airway inflammation.^{5,6}

Regional differences in asthma have been recently identified. Neutrophilic asthma remains the predominant form reported on the East Coast of the United States.⁷ Central and Western Mountain regions of North America have reported different phenotypes; often with an cosinophilic, mastocytic, or mixed phenotype.^{8,9} Seasonal variation is similarly reported, with winter exacerbations of neutrophilic asthma or summer pasture-related asthma.¹⁰

Allergic asthma has been well characterized in humans, with a strong mastocytic and eosinophilic component to the disease, however recent research has shown further molecular subtypes through T helper type (Th) 1, Th2, and Th17 pathways. These cytokine subtypes have been less well defined in horses.^{11,12} Th2 remains the predominant allergic asthma inflammatory pathway in humans.¹³ Eosinophilic asthma leads to inflammatory cytokine profiles that result in recruitment of mast cells and basophils, resulting in a mixed phenotypic profile, similar to that mixed profile found in horses.^{13,14} Type 2 cytokines predominate in these cases; including interleukin (IL)-4, IL-5, IL-13, and IL-18.^{14,15} This is in clear differentiation from the neutrophilic/non-type 2 inflammatory phenotype where IL-17 and IL-22 predominate, which is analogous to RAO in horses.¹⁴

The aim of the current study was to evaluate the clinical relevance of intradermal skin testing (IDT), environmental air quality, and serum cytokine biomarkers in the presence and severity of equine asthma in horses in Texas.

### Materials and Methods

### Study Design and Animals

This study was a multi-site, cohort study in horses with suspected asthma. A total of 52 horses with clinical signs or history consistent with equine asthma were enrolled. Horses were enrolled from four locations in West and Central Texas. IACUC approval was obtained prior to the conduct of the study.

### Eligibility Criteria

Horses aged 3-20 years of age were eligible for screening. All included horses were required to have either clinical signs and/or history consistent with equine asthma and no evidence of infectious respiratory disease. Horses were evaluated by physical examination, clinical pathology, and thoracic ultrasound for eligibility.

### Study Timeline

FIG. 19 is a flowchart that illustrates a study timeline.

Horses enrolled in the study were evaluated by clinical pathology (complete blood count, serum amyloid A, biochemistry panel) and a bronchoalveolar lavage (BAL) was performed. Blood was collected for analysis of 23 inflammatory cytokines by a multiplex profile.¹⁶ An environmental air monitor was used to measure aerosolized particles at each site for a minimum of one week.

Following evaluation of cytology (1-3 weeks after initial visit) all horses with a diagnosis of asthma were evaluated by intradermal skin testing (Greer Allergy Skin Testing) for 23 allergens common to Texas. Horses without a diagnosis of asthma were provided IDT at the discretion of the owner.

Bronchoalveolar Lavage (BAL). BALs were performed according to standard practice using 250 mL instilled volume of saline blindly into one lung lobe.^{17,18} Collected BALF was processed by cytocentrifugation and a 400 differential cell count was performed by a board-certified clinical pathologist.¹⁹ Diagnosis of asthma and cellular phenotype was determined according to current recommendations.¹⁸

Intradermal skin testing (IDT). Horses were clipped and prepped for intradermal skin testing on the lateral aspect of the neck. A 0.1 mL volume was injected for each of the 23 allergens, 1 positive control (histamine), and I negative control (saline) using a tuberculin syringe and each location was identified with permanent marker.3 Injection sites were measured by the maximum diameter 30 minutes post-injection. Saline ratio, histamine ratio, and severity were calculated for each lesion. A positive reaction was identified by both a saline ratio ≥1.2 and a histamine ratio ≥0.7.

Multiplex. Serum samples collected at the time of the BAL were analyzed by an equine multiplex profile (Milliplex MAP equine cytokine magnetic bead panel for 23 cytokines using a multiplex instrument.²⁰

Environmental Air Monitor. A DustTrak Aerosol Monitor (DustTrak DRX Desktop and DustTrak II aerosol) monitor and TSI GM460 handheld gas monitor were set in the horses' location to measure size segregated mass fraction concentrations (PMI, PM2.5, Respirable PM4, and PM10) over a wide concentration range (0.001-150 mg/m³). The monitor was enabled for real-time, direct-reading aerosol monitoring and analysis. Both monitors were placed in a horse barn 3 feet above the ground in the center of the aisle where occupied horse stalls were divided equally on each side of the monitor. For pasture-only housing the monitor was placed 3 feet above the ground approximately 10 feet from the water source.

Analysis. Summary statistics were reported for animal demographics, asthma diagnosis, IDT, and cytokine analysis. PM2.5 results were converted to air quality index (AQI) measurements based on recommendations from US EPA for 24 hour monitoring.²¹ IDT and cytokine results for different asthma subtypes were compared by Fisher's exact test. Multivariant correlations were evaluated for cytokines by BALF cytology cell counts. Horses with cytokine values below the limit of quantification were included in the analysis and a value of one half the lower limit of quantification was assigned to the horse. Spearman correlation was evaluated for cytokines only. Chi-square analysis was performed for AQI and asthma phenotype associations. Statistical analysis was performed using JMP PRO version 16.

Animal Demographics. Fifty-eight horses were screened for enrollment and 52 horses were included in the study. Horses ranged in age from 3-19 years of age. Twenty-four (46%) were geldings and 28 (54%) were mares. Quarter horses made up the largest breed category (n=25, 48%), followed by Thoroughbreds (n=13, 25%), Warmbloods (n=8, 15%), Irish Sport Horse (n=3, 6%), and one each of Appendix, Appaloosa, and mixed breed. Horses were sourced from four locations in Texas. The largest group were housed in stalls and on pasture (n=25, 48%), followed by stalls and dry lot (n=13, 25%), stall only (n=5, 10%), pasture only (n=4, 8%), and dry lot only (n=3, 6%).

Bronchoalveolar Lavage (BAL). BALF fluid was recovered from all enrolled horses. Cytologic diagnoses of asthma were made in 34 (65%) of enrolled horses and summarized in Table 1.

TABLE 1
Cytologic Phenotype of Asthma Diagnoses

| Cytologic Phenotype | N (%)* | |
|---|---|---|
| eosinophilic only | 6 | (17.6) |
| mastocytic only | 17 | (50) |
| neutrophilic only | 5 | (14.7) |
| eosinophilic/mastocytic | 4 | (11.8) |
| mastocytic/neutrophilic | 2 | (5.9) |

*percent of each phenotype is based on the 34 horses diagnosed with asthma

Intradermal skin testing (IDT). IDT was completed on 31 horses. 17 horses were tested for 23 allergens, while 14 horses were tested for 21 allergens. Results of IDT testing by asthma phenotype are summarized in Table 2. No statistical differences were found between asthma phenotypes and positive IDT results, and no correlations were identified between cytologic cell count and positive IDT results.

TABLE 2
Positive IDT Results by Asthma Phenotype

| | N (%) Positive IDT Results | | | | | | | |
|---|---|---|---|---|---|---|---|---|
| | Mastocytic | | Eosinophilic | | Neutrophilic | | Non- | |
| | Asthma | | Asthma | | Asthma | | Asthmatic | |
| Allergen | n | % | n | % | n | % | n | % |
| Pine Tree Mix | 7 | 39 | 3 | 43 | 1 | 20 | 1 | 17 |
| Box Elder | 5 | 28 | 3 | 43 | 1 | 20 | 1 | 17 |
| American Elm | 5 | 28 | 2 | 29 | 1 | 20 | 2 | 33 |
| Eastern | 9 | 50 | 3 | 43 | 0 | 0 | 2 | 33 |
| Cottonwood | | | | | | | | |
| Red Cedar | 8 | 44 | 4 | 57 | 2 | 40 | 3 | 50 |
| Mesquite | 9 | 50 | 5 | 71 | 1 | 20 | 3 | 50 |
| Firebush/ | 12 | 67 | 5 | 71 | 2 | 40 | 3 | 50 |
| Kochia | | | | | | | | |
| Russian Thistle | 13 | 72 | 5 | 71 | 3 | 60 | 4 | 67 |
| Ragweed mix | 9 | 50 | 3 | 43 | 2 | 40 | 2 | 33 |
| Mold mix | 10 | 56 | 4 | 57 | 2 | 40 | 3 | 50 |
| Drechslera | 6 | 33 | 1 | 14 | 0 | 0 | 3 | 50 |
| Spicifera | | | | | | | | |
| Grass Mix | 8 | 44 | 2 | 29 | 1 | 20 | 3 | 50 |
| Common Oats | 8 | 44 | 2 | 29 | 3 | 60 | 3 | 50 |
| Johnson Grass | 12 | 67 | 3 | 43 | 2 | 40 | 3 | 50 |
| Bermuda Grass | 9 | 50 | 4 | 57 | 1 | 20 | 3 | 50 |
| Cattle Epithelia | 12 | 67 | 5 | 71 | 3 | 60 | 3 | 50 |
| Mite mix | 15 | 83 | 7 | 100 | 5 | 100 | 6 | 100 |
| Horse Fly | 9 | 50 | 4 | 57 | 4 | 80 | 3 | 50 |
| Mosquito | 11 | 61 | 3 | 43 | 2 | 40 | 4 | 67 |
| Culicoides mix | 11 | 61 | 5 | 71 | 3 | 60 | 4 | 67 |
| House Fly | 12 | 67 | 4 | 57 | 3 | 60 | 4 | 67 |
| Birch mix* | 8 | 67 | 1 | 50 | 2 | 50 | 3 | 100 |
| Epicoccum | 8 | 67 | 1 | 50 | 3 | 75 | 2 | 67 |
| nigrum* | | | | | | | | |

Calculated percentages were from the total number of horses with the asthma phenotype. Mastocytic asthma n = 18; Eosinophilic asthma n = 7; Neutrophilic asthma n = 5; normal horses n = 6. Five horses had more than one asthma phenotype.
**4 animals were not tested for Birch and Epicoccum nigrum.

Cytokines. Serum cytokine analysis was completed on samples from 52 horses. Cytokines with 75% (n=39) or more of horses below the limit of detection were excluded from analysis. FGF-2, eotaxin, G-CSF, IL-la, GM-CSF, fractalkine, IL-13, IL-1b, IL-17a, IL-12, GRO, MCP-1, and RANTES were therefore excluded. Strong correlations (Spearman coefficient >0.8; p value <0.0001) were found for the following cytokine relationships: IL-2 and IL-5, IL-2 and IL-6, IL-2 and IFNy, IL-4 and IP-10, IL-5 and IL-6, IL-6 and IFNy, and IL-10 and TNFa. Cytokines included in the analysis are summarized in Table 3. No correlations were found between cytokine concentrations and BALF cytologic cell counts and no statistical difference was found between cytokine concentrations and asthma phenotype.

TABLE 3
Serum Cytokine Results by Asthma Phenotype

| | | | Mean ± SD | | | | |
|---|---|---|---|---|---|---|---|
| Cytokine | N | N | Eosinophilic | Mastocytic | Neutrophilic | Non- | |
| (pg/mL) | ALQ | BLQ | asthma | asthma | asthma | Asthmatic | All |
| IL-5 | 17 | 35 | 130 ± 235 | 106 ± 191 | 28 ± 2.7 | 66 ± 86 | 88 ± 161 |
| IL-18 | 37 | 14 | 161 ± 202 | 125 ± 142 | 72 ± 78 | 109 ± 94 | 120 ± 135 |
| IL-6 | 17 | 35 | 147 ± 304 | 122 ± 235 | 27 ± 0.5 | 75 ± 131 | 100 ± 206 |
| IL-2 | 16 | 36 | 37 ± 79 | 31 ± 62 | 7.4 ± 0.02 | 20 ± 32 | 26 ± 54 |
| IL-4 | 31 | 21 | 639 ± 940 | 647 ± 701 | 286 ± 245 | 549 ± 405 | 572 ± 631 |
| IFNy | 19 | 33 | 1373 ± 2941 | 1618 ± 2416 | 247 ± 6.6 | 992 ± 1699 | 1216 ± 2163 |
| IL-8 | 49 | 3 | 331 ± 107 | 326 ± 97 | 298 ± 95 | 287 ± 74 | 311 ± 91 |
| IP-10 | 39 | 11 | 55 ± 64 | 52 ± 32 | 45 ± 43 | 56 ± 39 | 53 ± 41 |
| IL-10 | 25 | 27 | 689 ± 1396 | 625 ± 1087 | 163 ± 151 | 456 ± 812 | 528 ± 995 |
| TNFa | 19 | 32 | 51 ± 101 | 41 ± 79 | 10 ± 3.9 | 32 ± 54 | 36 ± 71 |

ALQ—above limit of quantification; BLQ—below limit of quantification; n—number; IL—interleukin; IFNy—interferon gamma; IP-10—IFNy induced protein 10; TNFa—Tumor necrosis factor alpha.

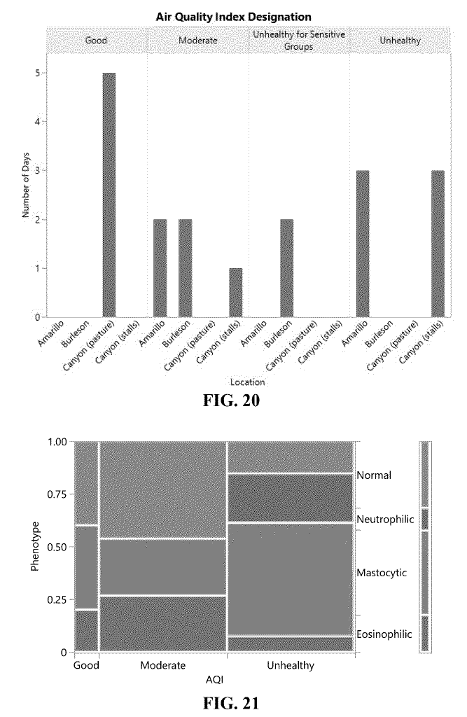

Environmental Monitoring. AQI values were calculated for each of the four locations housing horses enrolled in the study for four to five continuous days. One site (Canyon pasture) had good AQI for the entire evaluation period (FIG. 20). Two sites (Amarillo stalls, Canyon stalls) had unhealthy AQI for three of the evaluation days (FIG. 20).

A statistical difference by Chi-square analysis for asthma phenotype by AQI was identified. Of note, mastocytic asthma was overrepresented in facilities with an unhealthy AQI, and neutrophilic asthma was only identified at facilities with an unhealthy AQI (FIG. 21).

FIG. 20 is a graph that shows an Air Quality Index for Study Sites. FIG. 21 is a mosaic plot of asthma phenotype by AQI.

TABLE 4
Asthma Diagnosis by Study Site

| | Location | | | | | | | |
|---|---|---|---|---|---|---|---|---|
| | | | | | Canyon | | Canyon | |
| Asthma | Burleson | | Amarillo | | (stalls) | | (pasture) | |
| Diagnosis | n | % | n | % | n | % | n | % |
| Normal | 12 | 48.0% | 2 | 13.3% | 2 | 25.0% | 2 | 50.0% |
| Mild Asthma | 11 | 44.0% | 7 | 46.7% | 5 | 62.5% | 1 | 25.0% |
| Severe Asthma | 2 | 8.0% | 6 | 40.0% | 1 | 12.5% | 1 | 25.0% |
| All | 25 | 100% | 15 | 100% | 8 | 100% | 4 | 100% |

Measurement of inflammatory cytokines in both blood and BALF has been evaluated repeatedly.² Serum biomarkers have been potentially identified in neutrophilic asthma; including haptoglobin.²⁶ However, other studies have been unable to find an association between acute phase proteins and airway inflammation, particularly in horses that are not severely affected.²⁷ Other biomarkers with demonstrated roles in human and rodent models of asthma such as IL-4, IL-5, IL-13, and IFN-gamma have shown variable results in equine asthma phenotypes.² This study was comparable to other studies in the failure to identify a single biomarker for any specific asthma phenotype. However, general trends consistent with previous reports were identified with lower concentrations of IL-5, IL-6, IL-18, IL-4, IFN-gamma, IL-10, and TNFa in neutrophilic asthma in comparison to mastocytic and eosinophilic asthma, and correlations between inflammatory biomarkers.

Horses at study sites with worse stall-side AQI showed a higher percentage of horses with mastocytic asthma and were the only sites with horses with neutrophilic asthma. While the stall-side AQI is a more localized measure of air quality compared to regional AQI provided in weather reports, regional AQI has shown strong associations in humans with asthma prevalence.^{28,29} In humans, many of these association studies with AQI use population-based measures, such as city-wide emergency room presentations or longevity studies that are not available in veterinary medicine.^{29,30} However, stall-side AQI provides specific information on the hazards experienced by the animals and humans at the specific facility, which may be substantially worse than the regional AQI. In addition, the maximum AQI provides temporal data that would indicate specific instances of likely respiratory inflammation. On-farm implementation of this data facilitates mitigation strategies, including turnout of horses during stall cleaning or use of indoor arenas or use of respirators by staff during times of poor AQI.

In humans, peripheral cosinophilia, peak expiratory flow, and exhaled nitric oxide have clinical utility; while sputum cytology provides non-invasive clinical phenotyping.¹³ Applications of these techniques in veterinary medicine are either impractical or require specialized in-hospital equipment.

Therapies directed at allergic or type 2 inflammatory asthma in humans have been proposed as separate from those utilized for neutrophilic asthma and more targeted than conventional corticosteroids.¹⁴ Monoclonal antibodies directed at specific targets; such as IL-4/IL-13 pathways, IL-5, and thymic stromal lymphopoietin have shown patient improvements.^{13,14} Several have been approved for use in humans with cosinophilic asthma. In addition, janus kinase (JAK) inhibitors have been investigated with success in exploratory studies.^{14,31}

The system can be configured to evaluate the clinical utility of exhaled nitric oxide and other gases, gas temperature, and/or gas volume and to provide a diagnostic tool for cosinophilic and mastocytic asthma in animals. In accordance with the disclosed embodiments, if the system detects fractioned exhaled nitric oxide (FeNO) concentrations ≥20 ppb, there is a high likelihood the horse has asthma, and will be diagnosed with cosinophilic and/or mastocytic asthma if traditional diagnostic techniques were used. The level of particulates can also be detected.

For example, a normal level of nitric oxide is generally less than 20 parts per billion. Anything above this threshold can be considered pathologic. Likewise, any level above 20 parts per billion would, with high probability, be indicative of allergic asthma.

It should be appreciated that in certain embodiments, an environmental monitor and chemical sensor can be integrated into the system, e.g., into a single housing exposed to the inhalation and/or exhalation of the animal. For example, one or more environmental monitors can be configured to measure environmental loads of particulates of less than 2.5 microns. One or more chemical sensors can be housed inside the environmental monitor and is configured to measure noxious chemical levels of NO, NH₃, CO, SO₂, O₃ and the like. These monitors can be configured to vacuum air samples and process them in real time. The combined environmental sensor and chemical sensor can also be directly mounted to the halter on the horse, on, in, or within the mask/muzzle, in order to obtain noxious chemical levels near the animal's head (i.e., nostrils for inhalation).

In an embodiment, the system further comprises a processing system comprising at least one processor and a storage device communicatively coupled to the at least one processor, the storage device storing instructions which, when executed by the at least one processor, cause the at least one processor to perform operations comprising: receiving indicia data from at least one of the sensor, the environmental sensor, and the chemical sensor; analyzing the environmental data; and determining if the data is indicative of asthma.

A computer system can include an analysis module used to combine data from one or more of the monitors. This allows the system to assess each individual animal and/or the overall environment that the horse is experiencing. If the system identifies an animal with increased nitric oxide measurements, a medical exam can be scheduled. Asthmatic animals that are diagnosed with asthma can then be monitored with the nitric oxide monitor vs having a repeat bronchoalveolar lavage (BAL) procedure. Successfully treated asthmatics will have normal nitric oxide measurements. Asthmatic horses with uncontrolled asthma will continue to have increased nitric oxide measurements. Data can also be used to drive development of treatment pathways or protocols based on the collected data.

In the disclosed embodiments, nitric oxide levels of less than 20 ppb are considered in a normal range. For particulates, fewer identifiable particulates is always better, however, identification of particulates that are less than 10 microns in size may be indicative of a higher risk environment since these smaller particulates can more readily enter the lungs, and identification of particulates that are less than 2.5 microns in size may indicative of a higher risk environment since these smaller particles can more readily enter the bloodstream. The system can include a measurement on the air quality index (AQI) which can include ozone, particle pollution, sulfur dioxide, nitrogen dioxide, and carbon monoxide. An AQI above 100 is considered unhealthy for asthmatic animals. Even an AQI ranging from 50-100 can exacerbate symptoms of asthma.

As such, in certain embodiments, readings from the system can be provided to the analysis module, which can generate an average AQI. This information can be used to determine if the animal's environment is creating risk of airway inflammation. If further testing indicates animals within a proximate environment that generates particulates or other chemicals that stress the respiratory system, have unusually high occurrences of asthma, the environmental data may suggest that dust mitigation, allergen control, etc., may be useful in reducing airway inflammation.

The data can also be used to evaluate ventilation in the nearby environment, as well as how the environment changes during cleaning times, rising times, and the like. For example, an animal with asthma is best removed from its environment during cleaning because this time usually correlates with high levels of particulate matter in the air. An asthmatic animal will be more sensitive to high levels of particulate matter in the air. By removing the animal from the barn during cleaning time, it may help decrease the chances of a flare up.

In an embodiment, data collected by the system can be used for laboratory assessments. Exhaled nitric oxide can be assessed patient-side using an embodiment of the nitric oxide system to ensure sufficient exhalation pressure from the animal. If the system indicates elevated nitric oxide levels, Bronchoalveolar lavage fluid can be cytocentrifuged and cytological evaluation of stained slides will be completed by a clinical pathologist. Based on the results the animal can be diagnosed with asthma. Animals can be diagnosed as normal, mild to moderate asthma, or severe asthma. Asthma diagnosis will be further defined by the elevated cellular phenotype (e.g., neutrophilic, cosinophilic, mastocytic). Generally, neutrophil levels greater than 20% is indicative of severe asthma; cosinophil levels in the range of 1%-5%, or a mastocytes level in the range of 2%-5% is considered mild asthma. Eosinophils and mastocytes levels of 5% are moderate. Neutrophils levels between 10-12% are considered to be moderate. A normal animal will have none of these inflammatory cells, or at least no cosinophils and mastocytes, and less than 5% of neutrophils levels. For animals with mastocytic and/or cosinophilic asthma, they may be treated by, e.g., administering short-acting beta2-adrenoceptor agonists (SABAs), long-acting beta-adrenoceptor agonists (LABA), anticholinergic medications, Leukotriene receptor antagonists, Mast cell stabilizers, theophyllines, Macrolide antibiotics, adrenergic agonists, corticosteroids, or janus kinase inhibitors, such as Abrocitinib; Baricitinib; Delgocitinib (topical); Deucravacitinib; Fedratinib; Filgotinib; Momclotinib; Oclacitinib; Pacritinib; Peficitinib; Ritlecitinib; Ruxolitinib (oral); Ruxolitinib (topical); Tofacitinib, or Upadacitinib.

A processor can execute programming for implementing parts of the methods and systems disclosed herein. A computing device in the form of a computer configured to interface with controllers, peripheral devices, and other elements disclosed herein may include one or more processing units, memory, removable storage, and non-removable storage. Memory may include volatile memory and non-volatile memory. A processor may form part of a computer may include or have access to a computing environment that includes a variety of transitory and non-transitory computer-readable media such as volatile memory and non-volatile memory, removable storage and non-removable storage. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions, as well as data including image data.

The computer may include, or have access to, a computing environment that includes input, output, and a communication connection. The computer may operate in a networked environment using a communication connection to connect to one or more remote computers, remote sensors and/or controllers, detection devices, hand-held devices, multi-function devices (MFDs), speakers, mobile devices, tablet devices, mobile phones, Smartphone, or other such devices. The remote computer may also include a personal computer (PC), server, router, network PC, RFID enabled device, a peer device or other common network node, or the like. The communication connection may include a Local Area Network (LAN), a Wide Area Network (WAN), Bluetooth connection, wifi, or other networks.

An output is most commonly provided as a computer monitor, but may include any output device. An output and/or input may include a data collection apparatus associated with computer system. In addition, input, which commonly includes a computer keyboard and/or pointing device such as a computer mouse, computer track pad, or the like, allows a user to input instructions to computer system. A user interface can be provided using output and input. Output may function as a display for displaying data and information for a user, and for interactively displaying a graphical user interface (GUI).

Note that the term “GUI” generally refers to a type of environment that represents programs, files, options, and so forth by means of graphically displayed icons, menus, and dialog boxes on a computer monitor screen. A user can interact with the GUI to select and activate such options by directly touching the screen and/or pointing and clicking with a user input device such as, for example, a pointing device such as a mouse, and/or with a keyboard. A particular item can function in the same manner to the user in all applications because the GUI provides standard software routines (e.g., program module or node) to handle these elements and report the user's actions. The GUI can further be used to display the electronic service image frames as discussed below.

Computer-readable instructions, for example, program module or node, which can be representative of other modules or nodes described herein, are stored on a computer-readable medium and are executable by the processing unit of computer. Program module or node may include a computer application. A hard drive, CD-ROM, RAM, Flash Memory, and a USB drive are just some examples of articles including a computer-readable medium.

It is contemplated that any embodiment discussed in this specification can be implemented with respect to any method, kit, reagent, or composition of the invention, and vice versa. Furthermore, compositions of the invention can be used to achieve methods of the invention. It will be understood that particular embodiments described herein are shown by way of

illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Gerber V. Genetics of Equine Respiratory Disease. Veterinary Clinics: Equine Practice 2020; 36:243-253.
2. Simões J, Batista M, Tilley P. The immune mechanisms of severe equine asthma—current understanding and what is missing. Animals 2022; 12:744.
3. Lo Feudo C M, Stucchi L, Alberti E, et al. Intradermal testing results in horses affected by mild-moderate and severe equine asthma. Animals 2021; 11:2086.
4. Klier J, Lindner D, Reese S, et al. Comparison of four different allergy tests in equine asthma affected horses and allergen inhalation provocation test. Journal of Equine Veterinary Science 2021; 102:103433.
5. Pirie R, Collie D, Dixon P, et al. Inhaled endotoxin and organic dust particulates have synergistic proinflammatory effects in equine heaves (organic dust-induced asthma). Clinical & Experimental Allergy 2003; 33:676-683.
6. Wheeler EF, Diehl N K, Zajaczkowski J, et al. Particulate matter characterization in equestrian riding arenas. Transactions of the ASABE 2006; 49:1529-1538.
7. Mainguy-Seers S, Lavoie J P. Glucocorticoid treatment in horses with asthma: A narrative review. Journal of Veterinary Internal Medicine 2021; 35:2045-2057.
8. Léguillette R, Steinmann M, Bond S, et al. Tracheobronchoscopic assessment of exercise-induced pulmonary hemorrhage and airway inflammation in barrel racing horses. Journal of Veterinary Internal Medicine 2016; 30:1327-1332.
9. Sundman E A, Chigerwe M, Hunyadi L M. A retrospective study of bronchoalveolar lavage fluid analysis in barrel racing horses with exercise-induced pulmonary hemorrhage and asthma in Texas from 2016 to 2018. Research in Veterinary Science 2020; 132:338-341.
10. Sad E P, Hess T M, Santos H A, et al. Molecular and Cellular Evaluation of Horses With Summer Pasture Associated Asthma Syndrome. Journal of Equine Veterinary Science 2023; 131:104928.
11. Denning D W, Pashley C, Hartl D, et al. Fungal allergy in asthma-state of the art and research needs. Clinical and Translational Allergy 2014; 4:1-23.
12. van Tilburg Bernardes E, Gutierrez M W, Arrieta M-C. The fungal microbiome and asthma. Frontiers in Cellular and Infection Microbiology 2020; 10.
13. Oppenheimer J, Hoyte FC, Phipatanakul W, et al. Allergic and eosinophilic asthma in the era of biomarkers and biologics: similarities, differences and misconceptions. Annals of Allergy, Asthma & Immunology 2022; 129:169-180.
14. Matucci A, Vivarelli E, Nencini F, et al. Strategies targeting type 2 inflammation: from monoclonal antibodies to JAK-inhibitors. Biomedicines 2021; 9:1497.
15. Xu M H, Yuan F L, Wang S J, et al. Association of interleukin-18 and asthma. Inflammation 2017; 40:324-327.
16. Kang H, Lee G K C, Bienzle D, et al. Equine alveolar macrophages and monocyte-derived macrophages respond differently to an inflammatory stimulus. Plos One 2023; 18: e0282738.
17. Mazan M R, Hoffman A M. Clinical techniques for diagnosis of inflammatory airway disease in the horse. Clinical Techniques in Equine Practice 2003; 2:238-257.
18. Couëtil L, Cardwell J, Gerber V, et al. Inflammatory airway disease of horses-revised consensus statement. Journal of Veterinary Internal Medicine 2016; 30:503-515.
19. Pickles K, Pirie R, Rhind S, et al. Cytological analysis of equine bronchoalveolar lavage fluid. Part 2: comparison of smear and cytocentrifuged preparations. Equine Veterinary Journal 2002; 34:292-296.
20. Brown KA, Davidson E J, Johnson A L, et al. Inflammatory cytokines in horses with cervical articular process joint osteoarthritis on standing cone beam computed tomography. Equine Veterinary Journal 2021; 53:944-954.
21. Agency UEP. Technical Assistance Document for the Reporting of Daily Air Quality—the Air Quality Index (AQI). In. Research Triangle Park, NC: Air Quality Assessment Division; 2018.
22. van Damme C M, van den Broek J, Sloet van Oldruitenborgh-Oosterbaan M M. Discrepancies in the bilateral intradermal test and serum tests in atopic horses. Veterinary Dermatology 2020; 31:390-e104.
23. Evans A, Paradis M, O'Callaghan M. Intradermal testing of horses with chronic obstructive pulmonary disease and recurrent urticaria. American Journal of Veterinary Research 1992; 53:203-208.
24. Jose-Cunilleras E, Kohn C W, Hillier A, et al. Intradermal testing in healthy horses and horses with chronic obstructive pulmonary disease, recurrent urticaria, or allergic dermatitis. Journal of the American Veterinary Medical Association 2001; 219:1115-1121.
25. Lorch G, Hillier A, Kwochka K W, et al. Results of intradermal tests in horses without atopy and horses with chronic obstructive pulmonary disease. American Journal of Veterinary Research 2001; 62:389-397.
26. Gy C, Leclere M, Vargas A, et al. Investigation of blood biomarkers for the diagnosis of mild to moderate asthma in horses. Journal of Veterinary Internal Medicine 2019; 33:1789-1795.
27. Leclere M, Lavoie-Lamoureux A, Lavoie J P. Acute phase proteins in racehorses with inflammatory airway disease. Journal of Veterinary Internal Medicine 2015; 29:940-945.
28. To T, Shen S, Atenafu E G, et al. The air quality health index and asthma morbidity: a population-based study. Environmental Health Perspectives 2013; 121:46-52.
29. Rosser F, Han Y-Y, Rothenberger S D, et al. Air quality index and emergency department visits and hospitalizations for childhood asthma. Annals of the American Thoracic Society 2022; 19:1139-1148.
30. Myers J M B, Schauberger E, He H, et al. A Pediatric Asthma Risk Score to better predict asthma development in young children. Journal of Allergy and Clinical Immunology 2019; 143:1803-1810. e1802.
31. Braithwaite I E, Cai F, Tom J A, et al. Inhaled JAK inhibitor GDC-0214 reduces exhaled nitric oxide in patients with mild asthma: A randomized, controlled, proof-of-activity trial. Journal of Allergy and Clinical Immunology 2021; 148:783-789.

What is claimed is:

1. A Data-Logging Sensor Integration Block (DLSIB) system for real-time respiratory data for use with humans and animals comprising:
at least one chamber comprising an interior, an inlet and outlet for air, wherein two or more sensors are in fluid communication with the interior of the at least one chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor, an SpO₂ sensor, or a PaCO₂ sensor; and
a processor connected to each of the two or more sensors, wherein the DLSIB system measures in real-time or internally records outputs from the two or more sensors for each breath of a subject.

2. The system of claim 1, wherein at least one of: a partial pressure of NO, O₂, and CO₂ during the breath, an integrated quantity of oxygen added to blood, and the integrated quantity of CO₂ removed from the blood during each breath, are determined.

3. The system of claim 1, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, or inhalation of soot from fires, volcanic smog (vog) from volcanic eruptions, dust from burn pits, and dust inside mining shafts.

4. The system of claim 1, wherein the DLSIB system detects physiological and physical obstructions that prevent optimal oxygen-carbon dioxide exchange at an alveolar level, to expedite diagnostic and therapeutic pulmonary hygienic intervention.

5. The system of claim 1, wherein the DLSIB system is connected between an air pump or respirator and a mask; is integral with a mask; or is integral with an air pump or respirator.

6. The system of claim 1, further comprising a display connected to the processor, wherein the display shows subject data in an aggregated or disaggregated graphic.

7. The system of claim 1, wherein the processor and two or more sensors are wired or wireless, and an input and output of the at least one chamber each connect to an input and an output hose or at least one chamber, respectively.

8. The system of claim 1, wherein the DLSIB system is configured for use with an animal, mammal, or human.

9. The system of claim 1, wherein the system comprises, consists essentially of, or consists of at least one of: the nitric oxide sensor, the nitrous oxide sensor, the oxygen sensor, the ozone sensor, the carbon dioxide sensor, the carbon monoxide sensor, the pressure sensor, the temperature sensor, the humidity sensor, the ammonia sensor, the mass flow rate sensor, the SpO₂ sensor, and the PaCO₂ sensor.

10. A method of obtaining real-time respiratory data with a device comprising:
providing a Data-Logging Sensor Integration Block (DLSIB) system capable of connecting to a respiratory system of a subject for obtaining the real-time respiratory data, the device comprising:
at least one chamber comprising an interior, an inlet and outlet for air, wherein two or more sensors are in fluid communication with the interior of the at least one chamber, wherein the two or more sensors are selected from a nitric oxide sensor, a nitrous oxide sensor, an oxygen sensor, an ozone sensor, a carbon dioxide sensor, a carbon monoxide sensor, a pressure sensor, a temperature sensor, a humidity sensor, an ammonia sensor, a mass flow rate sensor, an SpO₂ sensor, or a PaCO₂ sensor, and
a processor connected to each of the two or more sensors, and wherein the DLSIB system measures in real-time outputs from the two or more sensors for each breath of a subject; and
calculating the real-time respiratory data with the processor.

11. The method of claim 10, wherein at least one of: a partial pressure of NO, O₂, and CO₂ during the breath, an integrated quantity of oxygen added to blood, and the integrated quantity of CO₂ removed from the blood during each breath, are determined.

12. The method of claim 10, wherein the DLSIB system analyzes, displays, and reports subject data to enable a provider to expedite diagnostic and therapeutic decisions and medical evaluations in real-time selected from asthma, respiratory distress, chronic obstructive pulmonary disease, emphysema, bronchitis, viruses, spores, elevation hypoxia, or inhalation of soot from fires, vog from volcanic eruptions, dust from burn pits, and dust inside mining shafts.

13. The method of claim 10, wherein the DLSIB system detects physiological and physical obstructions that prevent oxygen-carbon dioxide exchange at an alveolar level, and for diagnostic and therapeutic pulmonary hygienic intervention.

14. The method of claim 10, wherein the DLSIB system is connected between an air pump or respirator and a mask; is integral with a mask; or is integral with an air pump or respirator.

15. The method of claim 10, further comprising providing a display connected to the processor, wherein the display shows subject data in an aggregated or disaggregated graphic.

16. The method of claim 10, wherein the processor and the two or more sensors are wired or wireless, an input and output of the at least one chamber each connect to an input and an output hose or at least one chamber, respectively.

17. The method of claim 10, wherein the DLSIB system is configured for use with an animal, mammal, or human.

18. The method of claim 10, wherein the system comprises, consists essentially of, or consists of: the nitric oxide sensor, the nitrous oxide sensor, the oxygen sensor, the ozone sensor, the carbon dioxide sensor, the carbon monoxide sensor, the pressure sensor, the temperature sensor, the humidity sensor, the ammonia sensor, the mass flow rate sensor, the SpO₂ sensor, and the PaCO₂ sensor.

19. A method of determining an effectiveness of a pulmonary therapy, the method comprising:
(a) measuring real-time respiratory data comprising:
providing a Data-Logging Sensor Integration Block (DLSIB) system capable of connecting to a subject for obtaining the real-time respiratory data, the DLSIB system comprising:
at least one chamber comprising an interior, an inlet and outlet for air, wherein two or more sensors are in fluid communication with the interior of the at least one chamber, wherein the two or more sensors are selected from a nitric oxide sensor, a nitrous oxide sensor, an oxygen sensor, an ozone sensor, a carbon dioxide sensor, a carbon dioxide sensor, a pressure sensor, a temperature sensor, a humidity sensor, an ammonia sensor, a mass flow rate sensor, an SpO₂ sensor, or a PaCO₂ sensor; and
a processor connected to each of the two or more sensors, and wherein the DLSIB system measures in real-time outputs from the two or more sensors for each breath of a subject; and
calculating the real-time respiratory data with the processor;
(b) administering a candidate drug to a first subset of the subjects, and a placebo to a second subset of the subjects;
(c) obtaining real-time respiratory data from the first and second subset of subjects;
(d) calculating a difference between the real-time respiratory data in the first and second subset of subject; and
(e) if real-time respiratory data differs between the first and second subset of subjects then calculating an effectiveness of the pulmonary therapy.

20. A method of measuring respiratory function, the method comprising:
connecting a Data-Logging Sensor Integration Block (DLSIB) system capable of connecting to a subject for obtaining real-time respiratory data, the device comprising:
a mask or at least one chamber comprising an interior, an inlet and outlet for air, wherein two or more sensors are in fluid communication with the interior of the mask or at least one chamber, wherein the two or more sensors are selected from a nitric oxide sensor, a nitrous oxide sensor, an oxygen sensor, an ozone sensor, a carbon dioxide sensor, a pressure sensor, a temperature sensor, a humidity sensor, an ammonia sensor, a mass flow rate sensor, an SpO₂ sensor, or a PaCO₂ sensor; and
a processor connected to each of the two or more sensors, and wherein the DLSIB system measures in real-time outputs from the two or more sensors for each breath of a subject; and
calculating the real-time respiratory data with the processor to determine respiratory function.
